(19) United States
(12) Patent Application Publication
(10) Pub. No.: US 2025/0154513 A1
(43) Pub. Date: May 15, 2025

(54) Applications of Streptococcus-derived Cas9 nucleases on minimal Adenine-rich PAM targets

(71) Applicant: Massachusetts Institute of Technology, Cambridge, MA (US)

(72) Inventors: Pranam Chatterjee, Cambridge, MA (US); Noah Michael Jakimo, Boston, MA (US); Joseph M. Jacobson, Newton, MA (US)

(73) Assignee: Massachusetts Institute of Technology, Cambridge, MA (US)

(21) Appl. No.: 18/927,790

(22) Filed: Oct. 25, 2024

Related U.S. Application Data
(63) Continuation of application No. 17/053,713, filed on Nov. 6, 2020, now abandoned.
(60) Provisional application No. 62/667,579, filed on May 6, 2018.

(51) Int. Cl. C12N 15/74; C12N 9/22; C12N 15/11

(52) U.S. Cl. CPC C12N 15/74; C12N 9/22; C12N 15/111; C12N 2310/20

(57) ABSTRACT

Applications of a Streptococcus Cas9 ortholog from Streptococcus macacae (Smac Cas9), possessing minimal adenine-rich PAM specificity, include an isolated Streptococcus macacae Cas9 protein or transgene expression thereof, a CRISPR-associated DNA endonuclease with PAM interacting domain amino acid sequences that are at least 80% identical to that of the isolated Streptococcus macacae Cas9 protein, and an isolated, engineered Streptococcus pyogenes Cas9 (Spy Cas9) protein with a PID as either the PID amino acid composition of the isolated Streptococcus macacae Cas9 (Smac Cas9) protein or of a CRISPR-associated DNA endonuclease with PID amino acid sequences that are at least 80% identical to that of the isolated Streptococcus macacae Cas9 protein. A method for altering expression of at least one gene product employs Streptococcus macacae Cas9 endonucleases in complex with guide RNA, for specific recognition and activity on a DNA target immediately upstream of either an “NAA” or “NA” or “NAAN” PAM sequence.

### RELATED APPLICATIONS

This application is a continuation of co-pending U.S. patent application Ser. No. 17/053,713, filed Nov. 6, 2020, which is a 371 of PCT International App. Ser. No. PCT/US2019/030958, filed May 6, 2019, which claims the benefit of U.S. Provisional Application Ser. No. 62/667,579, filed May 6, 2018, the entire disclosures of which are herein incorporated by reference.

### INCORPORATION BY REFERENCE OF SEQUENCE LISTING XML

This application contains a Sequence Listing XML submitted under the provisions of 37 CFR 1.831(a) and herein incorporated by reference. The Sequence Listing XML includes, in XML format, the following file:

| File name | Creation Date | Size in bytes |
|---|---|---|
| MIT1120.xml | Oct. 25, 2024 8:47 PM | 42000 |

### FIELD OF THE TECHNOLOGY

The present invention relates to genome editing and, in particular, to a Streptococcus pyogenes Cas9 ortholog having novel PAM specificity, along with variants and uses thereof.

### BACKGROUND

CRISPR-associated (Cas) DNA-endonucleases are remarkably effective tools for genome engineering, but have limited target ranges due to their protospacer adjacent motif (PAM) requirements. In particular, the RNA-guided DNA endonucleases (RGENs), such as Cas9 and Cas12a, have proven to be versatile tools for genome editing and regulation [Sander, J. D. & Joung, J. K., “CRISPR-Cas systems for editing, regulating and targeting genomes”, Nature Biotechnology, vol. 32, pages 347-355, 2014; Doudna, J. A. & Charpentier, “E. Genome editing. The new frontier of genome engineering with CRISPR-Cas9”, Science, vol. 346, page 1258096, 2014]. The range of targetable sequences is limited, however, by the need for a specific protospacer adjacent motif (PAM), which is determined by DNA-protein interactions, to immediately precede or follow the DNA sequence specified by a guide RNA (gRNA) [Mojica, F. J., et al., “Short motif sequences determine the targets of the prokaryotic CRISPR defense system”, Microbiology 155, 733-740 (2009); Shah, S. A., et al., “Protospacer recognition motifs: mixed identities and functional diversity”, RNA Biology 10, 891-899 (2013); Jinek, M. et al., “A programmable dual-RNA-guided DNA endonuclease in adaptive bacterial immunity”, Science 337, 816-821 (2012); Sternberg, S. H., et al., “DNA interrogation by the CRISPR RNA-guided endonuclease Cas9”, Nature 507, 62-67 (2014); Zetsche, B., et al., “Cpf1 is a Single RNA-Guided Endonuclease of a Class 2 CRISPR-Cas System”, Cell 163:3, 759-771 (2015)].

While biotechnologies based on RNA-guided CRISPR systems have enabled precise and programmable genomic interfacing [Komor, A. C., Badran, A. H. & Liu, D. R., “Crispr-based technologies for the manipulation of eukaryotic genomes”, Cell 168, 20-36 (2017)], CRISPR-associated (Cas) endonucleases are collectively restrained from localizing to any position along double-stranded DNA (dsDNA) due to its requirement for targets to neighbor a protospacer adajacent motif (PAM) [Mojica, F. J. M., Diez-Villasenor, C., Garcia-Martinez, J. & Almendros, C., “Short motif sequences determine the targets of the prokaryotic CRISPR defence system”, Microbiology (Reading, England) 155, 733-740 (2009); Sternberg, S. H., Redding, S., Jinek, M., Greene, E. C. & Doudna, J. A., “DNA interrogation by the CRISPR RNA-guided endonuclease Cas9”, Nature 507, 62-67 (2014); Leenay, R. T. & Beisel, C. L. Deciphering, communicating, and engineering the CRISPR PAM”, Journal of Molecular Biology 429, 177-191 (2017)]. Current gaps in the PAM sequences that Cas enzymes are known to recognize prevent access to numerous genomic positions for powerful methods like base editing, which can only operate on a narrow window of nucleotides at fixed distances from the PAM [Komor, A. C., Kim, Y. B., Packer, M. S., Zuris, J. A. & Liu, D. R., “Programmable editing of a target base in genomic DNA without double-stranded DNA cleavage”, Nature 533, 420-424 (2016)]. Many AT-rich regions, in particular, have been excluded from compelling CRISPR applications because previously reported endonucleases, such as Cas9 and Cas12a (formerly known as Cpf1), require targets to neighbor GC-content or more restrictive motifs, respectively [Zhang, M. et al., “Uncovering the essential genes of the human malaria parasite Plasmodium falciparum by saturation mutagenesis”, Science (New York, N.Y.) 360 (2018); Jinek, M. et al., “A programmable dual-RNA-guided DNA endonuclease in adaptive bacterial immunity”, Science (New York, N.Y.) 337, 816-821 (2012); Zetsche, B. et al., “Cpf1 is a single RNA-guided endonuclease of a class 2 CRISPR-Cas system”, Cell 163, 759-771 (2015)].

For example, the most widely used variant, Streptococcus pyogenes Cas9 (SpCas9), requires a minimal, guanine (G)-rich 5′-NGG-3′ motif downstream of its RNA-programmed DNA target [Jinek, M. et al., “A programmable dual-RNA-guided DNA endonuclease in adaptive bacterial immunity”, Science 337, 816-821 (2012)]. In applications that require targeting a precise position along DNA, the current sequence-limitation imposed by the small set of known PAM motifs has constrained the impact of synthetic genome engineering efforts [Mojica, F. J., et al., “Short motif sequences determine the targets of the prokaryotic CRISPR defense system”, Microbiology 155, 733-740 (2009); Jinek, M. et al., “A programmable dual-RNA-guided DNA endonuclease in adaptive bacterial immunity”, Science 337, 816-821 (2012); Zetsche, B., et al., “Cpf1 is a Single RNA-Guided Endonuclease of a Class 2 CRISPR-Cas System”, Cell 163:3, 759-771 (2015)].

To relax this constraint, additional Cas9 and Cas12a variants with distinct PAM motif requirements have been discovered in nature or engineered to diversify the space of targetable DNA sequences. Bioinformatics tools have been utilized to align Clustered Regularly Interspaced Short Palindromic Repeats (CRISPR) cassettes of numerous bacterial species with presumed protospacers in phage or other genomes. This mapping helps to infer and subsequently test PAM sequences of naturally-occurring orthologs that possess useful properties, such as decreased size [Ran, F. A. et al., “In vivo genome editing using Staphylococcus aureus Cas9”, Nature 520, 186-191 (2015); Kim, E. et al., “In vivo genome editing with a small Cas9 orthologue derived from Campylobacter jejuni”, Nature Communications 8, 14500 (2017)] and thermostability [Harrington, L. et al., “A thermostable Cas9 with increased lifetime in human plasma”, bioRxiv (2017)]. However, such analysis does not guarantee efficient activity, and must be followed by assays to validate PAMs. Alternatively, functionally efficient RGENs, such as SpCas9 and Acidaminococcus sp. Cas12a (AsCas12a), have been utilized as scaffolds for engineering to produce variants with altered PAM specificities [Kleinstiver, B. P. et al., “Engineered CRISPR-Cas9 nucleases with altered specificities”, Nature 523, 481-485 (2015); Gao, L., et al., “Engineered Cpf1 variants with altered specificities”, Nature Biotechnology 35, 789-792 (2017)], with measured success.

It has been well documented that the canonical 5′-NGG-3′ specificity of SpCas9 derives in part from its possession of two arginine residues critical for PAM binding [Anders, C. et al., “Structural basis of PAM-dependent target recognition by the Cas9 endonuclease”, Nature 513, 569-573 (2014); Luscombe, N. M., et al., “Amino acid-base interactions: a three-dimensional analysis of protein-DNA interactions at an atomic level”, Nucleic Acids Res. 29, 2860-2874 (2001)]. While arginine residues are commonly used by DNA-binding proteins to recognize guanines (G′s), binding to adenines (A′s) typically involves glutamine residues [Luscombe, N. M., et al., “Amino acid-base interactions: a three-dimensional analysis of protein-DNA interactions at an atomic level”, Nucleic Acids Res. 29, 2860-2874 (2001)]. For example, the Cas9 ortholog from Lactobacillus buchneri, which possesses glutamine residues at positions equivalent to that of the two critical arginine residues (1333 and 1335) of SpcCas9, has been predicted to bind the 5′-NAAAA-3′ PAM sequence [Briner, A. E., et al., “Lactobacillus buchneri genotyping on the basis of clustered regularly interspaced short palindromic repeat (CRISPR) locus diversity”, Appl. Environ. Microbiol. 80, 994-1001 (2014)].

### SUMMARY

In one aspect, the invention includes genome engineering applications of a novel Streptococcus Cas9 ortholog from Streptococcus macacae (Smac Cas9) and its engineered variants, possessing minimal adenine (A)-rich PAM specificity. Smac Cas9 is a closely related ortholog of Streptococcus pyogenes Cas9 (Spy Cas9) that also contains two glutamine residues at these two positions, thus potentially possessing A-rich PAM specificity. Furthermore, exploiting the N-terminal homology of Spy Cas9 in combination with the PID of Smac Cas9 enables a more minimal PAM sequence, with fewer bases of preference.

In one aspect, the invention includes a critical expansion of the targetable sequence space for a Type-IIA CRISPR-associated enzyme through identification of the natural 5′-NAA-3′ PAM specificity of a Streptococcus macacae Cas9 (Smac Cas9). Protein domains are further recombined between SmacCas9 and its well-established ortholog from Streptococcus pyogenes (SpyCas9), as well as an “increased” nucleolytic variant (iSpy Cas9), to achieve consistent mediation of gene modification and base editing. In a comparison to previously reported Cas9 and Cas12a enzymes, the present hybrids recognize all adenine dinucleotide PAM sequences and possess robust editing efficiency in human cells.

A homolog of Spy Cas9 in Streptococcus macacae with native 5′-NAAN-3′ PAM specificity has been identified. By leveraging the substantial background in the development and characterization of Spy Cas9, variants of Smac Cas9 have been engineered that maintain its minimal adenine dinucleotide PAM specificity and achieve suitable activity for mediating edits on chromosomes in human cells [Jiang, F. & Doudna, J. A., “Crispr-Cas9 structures and mechanisms”, Annual review of biophysics 46, 505-529 (2017)]. This sets the path for engineering enzymes like Spy-mac Cas9 with other desirable properties, control points, effectors, and activities [Hu, J. H. et al., “Evolved Cas9 variants with broad pam compatibility and high DNA specificity”, Nature 556, 57-63 (2018); Slaymaker, I. M. et al., “Rationally engineered Cas9 nucleases with improved specificity”, Science (New York, N.Y.) 351, 84-88 (2016); Holtzman, L. & Gersbach, C. A., “Editing the epigenome: Reshaping the genomic landscape”, Annual review of genomics and human genetics 19, 43-71 (2018); Gutschner, T., Haemmerle, M., Genovese, G., Draetta, G. F. & Chin, L., “Post-translational regulation of Cas9 during g1 enhances homology-directed repair”, Cell reports 14, 1555-1566 (2016)]. Spy-mac Cas9 can now open wide access to AT-content PAM sequences in the ever-growing list of genome engineering applications with Type-IIA CRISPR-Cas systems.

In one aspect, the invention includes an isolated Streptococcus macacae Cas9 (Smac Cas9) protein or transgene expression thereof. In another aspect, it includes a CRISPR-associated DNA endonuclease with PAM interacting domain (PID) amino acid sequences that are at least 80% identical to that of the isolated Streptococcus macacae Cas9 (Smac Cas9) protein. The CRISPR-associated DNA endonuclease may have a PAM specificity of “NAA” or “NA” or “NAAN”.

In another aspect, the invention includes an isolated, engineered Streptococcus pyogenes Cas9 (Spy Cas9) protein with its PID as either the PID amino acid composition of the isolated Streptococcus macacae Cas9 (Smac Cas9) protein or that of a CRISPR-associated DNA endonuclease with PAM interacting domain (PID) amino acid sequences that are at least 80% identical to that of the isolated Streptococcus macacae Cas9 (Smac Cas9) protein.

In yet another aspect, the invention includes a method for altering expression of at least one gene product by employing Streptococcus macacae Cas9 (Smac Cas9) endonucleases in complex with guide RNA, consisting of identical non-target-specific sequence to that of the guide RNA Smac Cas9, for specific recognition and activity on a DNA target immediately upstream of either an “NAA” or “NA” or “NAAN” PAM sequence.

In a further aspect, the invention includes a method of altering expression of at least one gene product comprising introducing, into a eukaryotic cell containing and expressing a DNA molecule having a target sequence and encoding the gene product, an engineered, non-naturally occurring CRISPR-Cas system comprising one or more vectors comprising (a) a regulatory element operable in a eukaryotic cell operably linked to at least one nucleotide sequence encoding a CRISPR system guide RNA that hybridizes with the target sequence, and (b) a second regulatory element operable in a eukaryotic cell operably linked to a nucleotide sequence encoding one or more of an isolated Streptococcus macacae Cas9 (Smac Cas9) protein or transgene expression thereof, a CRISPR-associated DNA endonuclease with PAM interacting domain (PID) amino acid sequences that are at least 80% identical to that of the isolated Streptococcus macacae Cas9 (Smac Cas9) protein, and an isolated, engineered Streptococcus pyogenes Cas9 (Spy Cas9) protein with its PID as either the PID amino acid composition of the isolated Streptococcus macacae Cas9 (Smac Cas9) protein or that of a CRISPR-associated DNA endonuclease with PAM interacting domain (PID) amino acid sequences that are at least 80% identical to that of the isolated Streptococcus macacae Cas9 (Smac Cas9) protein, wherein components (a) and (b) are located on same or different vectors of the system, whereby the guide RNA targets the target sequence and one or more of the proteins cleave the DNA molecule, whereby expression of the at least one gene product is altered, and wherein the proteins and the guide RNA do not naturally occur together.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 is a comparison of the amino acid sequence alignment of Streptococcus pyogenes Cas9 (Spy Cas9) [SEQ ID No: 31], its “QQR” variant [SEQ ID No: 32], and the ortholog Streptococcus macacae (Smac Cas9) [SEQ ID No: 33].

FIG. 2 depicts the domain organization of Spy Cas9, juxtaposed over a color-coded structure of RNA-guided, target-bound Spy Cas9.

FIG. 3 depicts the sequence alignment for selected orthologs of interest that substitute at least one critical PAM-contacting arginine residue.

FIG. 4 is a generated sequence logo from input with putative PAM sequences found in Streptococcus phage and associated with close Smac Cas9 homologs.

FIGS. 5A-C depict annotated CRISPR cassettes obtained from the genomes corresponding to Smac (FIG. 5A), Smut1 (FIG. 5B), and Smut2 (FIG. 5C) orthologs that substitute glutamine for both PAM-contacting arginine residues.

FIG. 6 depicts mappings of CRISPR cassette spacers to their putative target source for listed crRNA.

FIG. 7 depicts sequencing chromatograms demonstrating the PAM-SCANR-based enrichment of variant-recognizing PAM sequences from a 5′-NNNNNNNN-3′ library for Spy dCas9 and Spy-mac dCas9.

FIG. 8 depicts chromatograms representing the PAM-SCANR based enrichment of variant-recognizing PAM sequences from a 5′-NNNNNNNN-3′ library for Spy-Ortholog Hybrid dCas9.

FIG. 9 is an SDS-PAGE gel image of Spy-mac Cas9 after purification by affinity chromatography.

FIG. 10 depicts SYBR-stained agarose gels showing in vitro digestion of 10 nM 5′-NAAN-3′ substrates.

FIG. 11 is a plot of timecourse measurements of target DNA substrate cleavage for Smac Cas9 and Spy-mac Cas9.

FIG. 12 is a plot of DNA substrate cleavage plotted as a function of 0.25:1, 1:1, and 4:1 molar ratios of ribonucleoprotein to target for wild-type Spy Cas9 and hybrid Spy-mac Cas9.

FIG. 13 depicts SYBR-stained agarose gels for in vitro digestion reactions that assay dependencies on crRNA spacer length.

FIG. 14 depicts SYBR-stained agarose gels for in vitro digestion reactions that assay dependencies on tracrRNA sequence origin.

FIG. 15 depicts sequence alignment of tracrRNA from S. pyogenes and S. mutans highlighted in a color code that reflects the base-pairing in their duplex gRNA secondary structure.

FIG. 16 depicts the duplex gRNA secondary structure of S. pyogenes and S. mutans, with base-pairing highlighted according to FIG. 15.

FIG. 17 depicts SYBR-stained agarose gels for in vitro digestion reactions that assay dependencies on positions 5-8 in the PAM sequence.

FIG. 18 depicts SYBR-stained agarose gels for in vitro digestion reactions that assay dependencies on increments to the distribution of adenine content in positions 1-5 in the PAM sequence.

FIG. 19 depicts detection of genomic modification in SYBR-stained agarose gels for T7EI digests upon targeting a single PAM site with combinations of wild-type plus hybrid variants of Cas9 and guide scaffold (tracrRNA sequence) from S. pyogenes and S. macacae.

FIG. 20 depicts SYBR-stained agarose gels showing a diversity of PAM sequences with the wild-type and engineered variants that include the Smac Cas9 PI domain.

FIG. 21 is a schematic diagram for matching Cas9 and Cas12a guides in a manner that enforces their recognition of the same PAM sequence and therefore facilities their comparison.

FIGS. 22A and 22B are dot plots of absolute (FIG. 22A) and relative (FIG. 22B) gene modification efficiency in HEK293T cells by Cas9 and Cas12a variants targeting common PAM sequences located in the VEGFA gene.

FIG. 23 is a chromatogram depicting a genomic base editing demonstration for the targeted conversion of cytosines to thymines with Spy-mac nCas9-BE3.

FIG. 24 depicts example results from T7E1 indel analysis, demonstrating effective cleavage on 5′-NAA-3′ targets with a variety of base combinations at positions 1 and 4 in the PAM sequence of the VEGFA gene.

### DETAILED DESCRIPTION

The invention includes genome engineering applications of a novel Streptococcus Cas9 ortholog, derived from Streptococcus macacae (Smac Cas9), and its engineered variants, possessing minimal adenine-rich PAM specificity. In one aspect, the invention is an addition to the family of CRISPR-Cas9 systems, repurposed for genome engineering and regulation applications. The invention further comprises Smac Cas9-variants engineered to possess mutations enabling the reduction of PAM specificity to 5′-NA-3′ through both random and rational manipulation of its open reading frame (ORF).

Specifically, the invention comprises the usage of either the Streptococcus macacae (Smac Cas9) endonuclease or the PAM-interacting domain of Smac Cas9 grafted onto the homologous N-terminal domain of Spy Cas9 (Spy-mac Cas9), in complex with guide RNA, to enable specific recognition and activity on a DNA target immediately upstream of either an 5′-NAA-3′ or 5′-NA-3′ PAM sequence, promoting new flexibility in target selection. Smac Cas9 is a closely related ortholog of Spy Cas9 that contains two glutamine residues at these two positions. Exploiting the N-terminal homology of Spy Cas9 in combination with the PID of Smac Cas9 enables a more minimal PAM sequence, with fewer bases of preference.

A homolog of Spy Cas9 in Streptococcus macacae with native 5′-NAAN-3′ PAM specificity has been identified. By leveraging the substantial background in the development and characterization of Spy Cas9, variants of Smac Cas9 that maintain its minimal adenine dinucleotide PAM specificity were engineered and suitable activity for mediating edits on chromosomes in human cells was achieved [Jiang, F. & Doudna, J. A., “Crispr-cas9 structures and mechanisms”, Annual review of biophysics 46, 505-529 (2017)]. This finding sets the path for engineering enzymes like Spy-mac Cas9 with other desirable properties, control points, effectors, and activities [Hu, J. H. et al., “Evolved Cas9 variants with broad pam compatibility and high DNA specificity”, Nature 556, 57-63 (2018); Slaymaker, I. M. et al., “Rationally engineered Cas9 nucleases with improved specificity”, Science (New York, N.Y.) 351, 84-88 (2016); Holtzman, L. & Gersbach, C. A., “Editing the epigenome: Reshaping the genomic landscape”, Annual review of genomics and human genetics 19, 43-71 (2018); Gutschner, T., Haemmerle, M., Genovese, G., Draetta, G. F. & Chin, L., “Post-translational regulation of Cas9 during g1 enhances homology-directed repair”, Cell reports 14, 1555-1566 (2016)]. Spy-mac Cas9 can now open wide access to AT-content PAM sequences in the ever-growing list of genome engineering applications with Type-IIA CRISPR-Cas systems.

The Cas9 ortholog derived from Streptococcus macacae NCTC 11558 can recognize a short 5′-NAA-3′ PAM [Richards, V. P. et al., “Phylogenomics and the dynamic genome evolution of the genus Streptococcus”, Genome biology and evolution 6, 741-753 (2014)]. These sequences constitute 18.6% of the human genome, making adjacent adenines the most abundant dinucleotide. The importance of this alternative PAM recognition for a Cas9 is reinforced by recent work which demonstrates that, while many Cas12a orthologs have AT-rich PAM sequences and are highly accurate nucleases on dsDNA, they will also indiscriminately digest single-stranded DNA (ssDNA) when bound to their targets [Chen, J. S. et al., “CRISPR-Cas12a target binding unleashes indiscriminate single-stranded dnase activity”, Science (New York, N.Y.) 360, 436-439 (2018); Kleinstiver, B. P. et al., “Genome-wide specificities of CRISPR-Cas Cpf1 nucleases in human cells”, Nature biotechnology 34, 869-874 (2016)]. Such collateral activity may introduce unwanted risks around partially unpaired chromosomal structures, such as transcription bubbles, R-loops, and replication forks. Engineered nucleases were derived from Smac Cas9 and their novel specificity and utility were characterized by means of transcriptional repression in bacterial culture, in vitro digestion reactions, and both gene and base editing in a human cell line.

To modify the ancestral 5′-NGG-3′ PAM specificity of Spy Cas9, previous works have employed directed evolution (e.g., “VQR”, “EQR”, and “VRER” variants) and rational design informed by crystal structure (e.g., “QQR” and “NG” variants) [Anders, C., Bargsten, K. & Jinek, M., “Structural plasticity of PAM recognition by engineered variants of the RNA-guided endonuclease Cas9”, Molecular Cell 61, 895-902 (2016); Kleinstiver, B. P. et al., “Engineered CRISPR-Cas9 nucleases with altered PAM specificities”, Nature 523, 481-485 (2015); Kleinstiver, B. P. et al., “Broadening the targeting range of Staphylococcus aureus CRISPR-Cas9 by modifying PAM recognition”, Nature Biotechnology 33, 1293-1298 (2015); Nishimasu, H. et al., “Engineered CRISPR-Cas9 nuclease with expanded targeting space”, Science (New York, N.Y.) (2018)]. These works focused on the PAM-contacting arginine residues R1333 and R1335 that abolish function when exclusively mutated. While those studies identified compensatory mutations resulting in altered PAM specificity, the Cas9 variants that they produced maintained a guanine preference in at least one position of the PAM sequence for reported in vivo editing. The present invention eliminates such GC-content pre-requisites via a custom bioinformatics-driven workflow that mines natural PAM diversity in the Streptococcus genus. Using that workflow, Smac Cas9 was identified as having the potential to bear novel PAM specificity upon aligning 115 orthologs of Spy Cas9 from UniProt (limited to those with greater than a 70% pairwise BLOSSOM62 score).

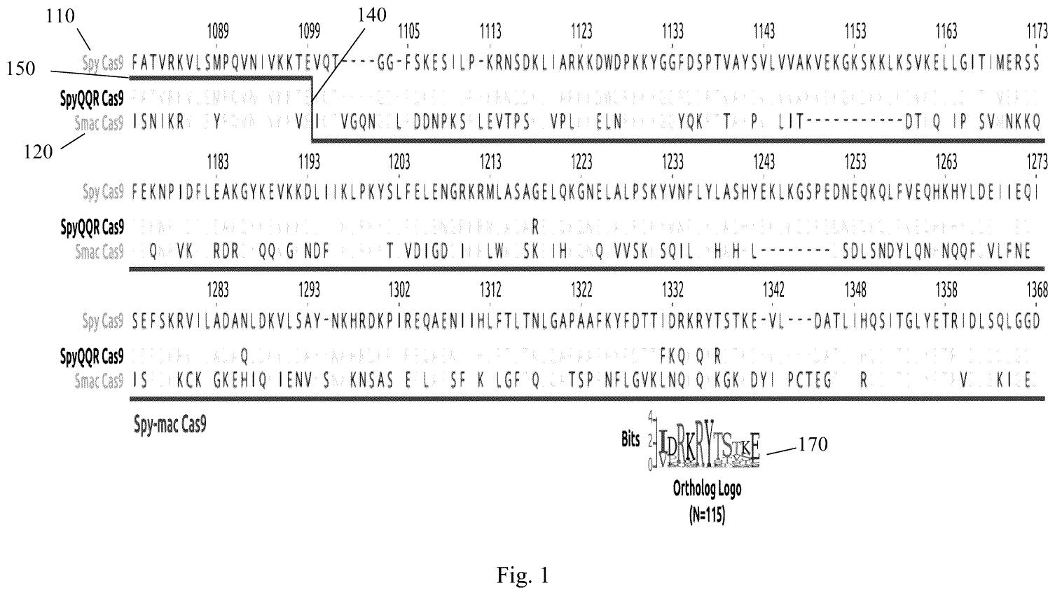

From the alignment, it was found that Smac Cas9 was one of two close homologs, along with a Streptococcus mutans B112SM-A Cas9 (Smut Cas9), with divergence at both of the positions aligned to the otherwise highly-conserved PAM-contacting arginines. FIG. 1 depicts the sequence alignment (Genewiz software) of Spy Cas9 110, its “QQR” variant, and Smac Cas9 120. The step 140 in underlining line 150 marks the joining of Spy Cas9 110 and Smac Cas9 130 to construct a Spy-mac Cas9 hybrid. Sequence logo 170 (Weblogo online tool) immediately below the alignment depicts the conservation at 11 positions around the PAM-contacting arginines of Spy Cas9.

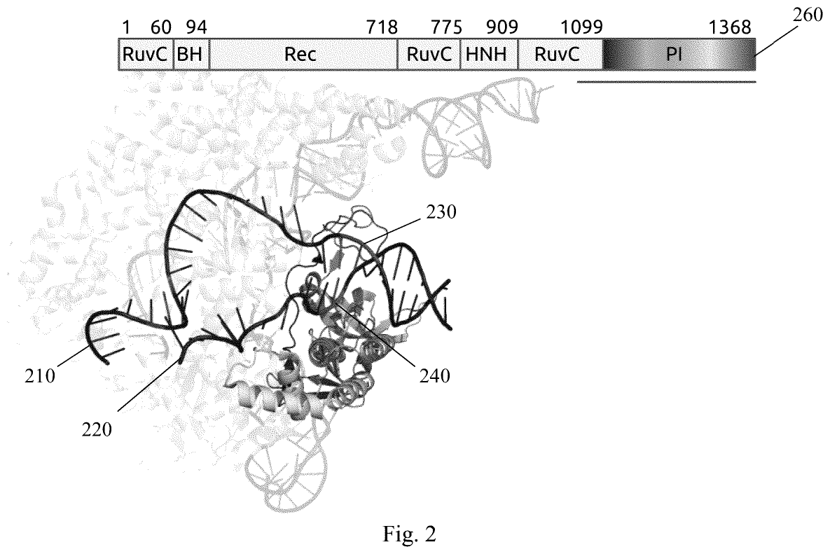

FIG. 2 depicts the domain organization of Spy Cas9 juxtaposed over a color-coded structure of RNA-guided, target-bound Spy Cas9 (PDB ID 5F9R). The two DNA strands 210, 220 are shown having segments 230, 240 corresponding to the PAM. Map 260 is used for labeling the Cas9 PI domain and guide spacer sequence to highlight structures that confer sequence specificity and the prevalence of intra-domain contacts within the PI [Jiang, F. et al., “Structures of a CRISPR-Cas9 r-loop complex primed for DNA cleavage”, Science (New York, N.Y.) 351, 867-871 (2016)].

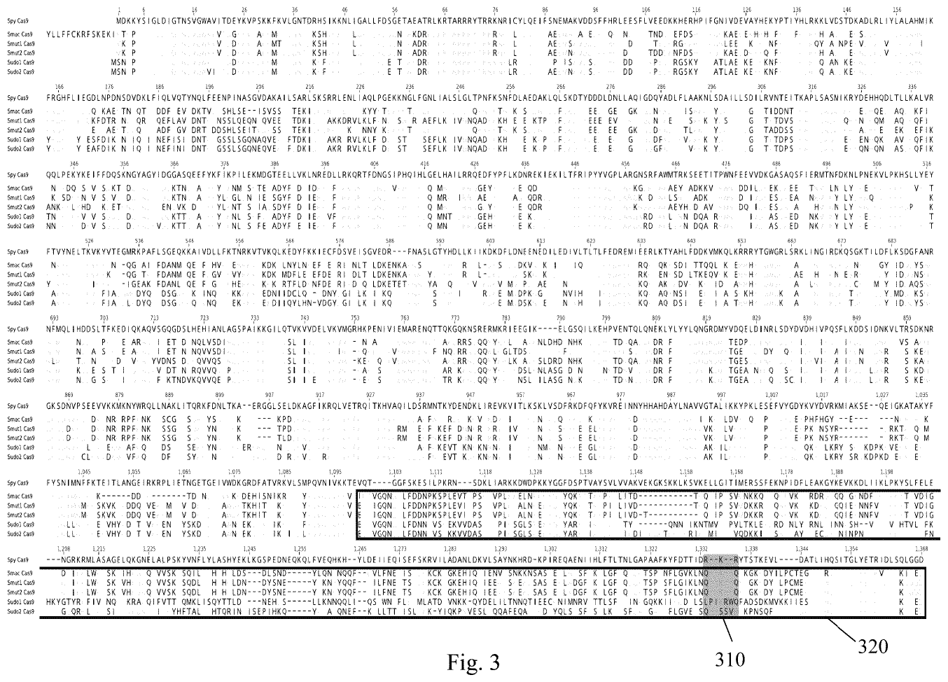

FIG. 3 depicts the sequence alignment (Genewiz software) for selected orthologs of interest that substitute at least one critical PAM-contacting arginine residue within region 310. Box 320 marks the C-terminal component grafted onto truncated Spy Cas9 to form dCas9 hybrids.

It was hypothesized that Smac Cas9 had naturally co-evolved the necessary compensatory mutations to gain new PAM recognition. A small sample size of 13 spacers from its corresponding genome's CRISPR cassette prevented confidently inferring the Smac Cas9 PAM in silico. However, the possibility for Smac Cas9 requiring less GC-content in its PAM was supported by sequence similarities to the “QQR” variant that has 5′-NAAG-3′ specificity, in addition to the AT-rich putative consensus PAM for phage-originating spacers in CRISPR cassettes associated with highly homologous Smut Cas9, which were identified with the aid of a computational pipeline called SPAMALOT [Chatterjee, P., Jakimo, N. & Jacobson, J. M., “Divergent PAM specificity of a highly-similar SpCas9 ortholog”, bioRxiv (2018)].

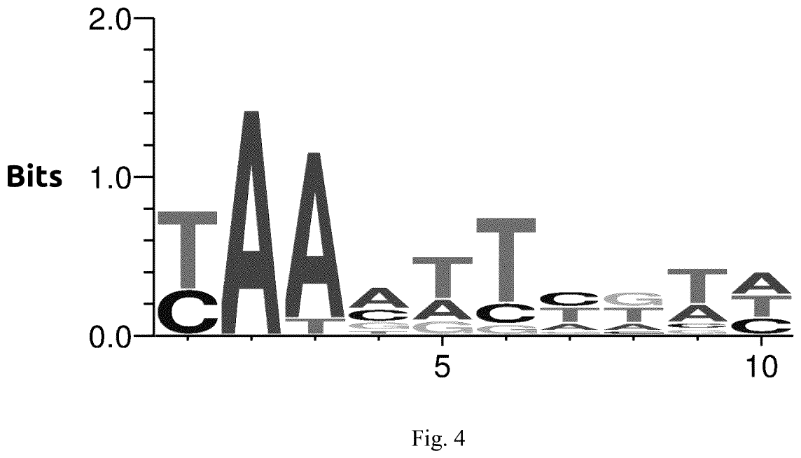

FIG. 4 is sequence logo generated online (WebLogo) that was input with putative PAM sequences found in Streptococcus phage and associated with close Smac Cas9 homologs. Table 1 lists the homology shared within and outside of box 320 of FIG. 3 to those regions in the corresponding Spy Cas9 and Smac Cas9 reference sequences.

TABLE 1

| Ortholog of Interest | % Agreement | % Agreement | % Agreement |
|---|---|---|---|
| NCBI Accession | to Spy1-1099 | to Spy1100-1368 | to Smac1100-1368 |
| Smac Cas9 WP_003079701 | 64.6 | 37.9 | — |
| Smut1 Cas9 BAQ19582 | 63.5 | 40.9 | 85.4 |
| Smut2 Cas9 WP_024784288 | 65.7 | 40.9 | 85.4 |
| Sudo1 Cas9 WP_049510439 | 64.4 | 30.2 | 39.9 |
| Sudo2 Cas9 WP_049538452 | 64.8 | 48.8 | 46.8 |

S = [symbol] treptococcus, py = [symbol] ogens, mac = [symbol] cacae, mut = [symbol] tans, udo = pse [symbol] pneumoniae

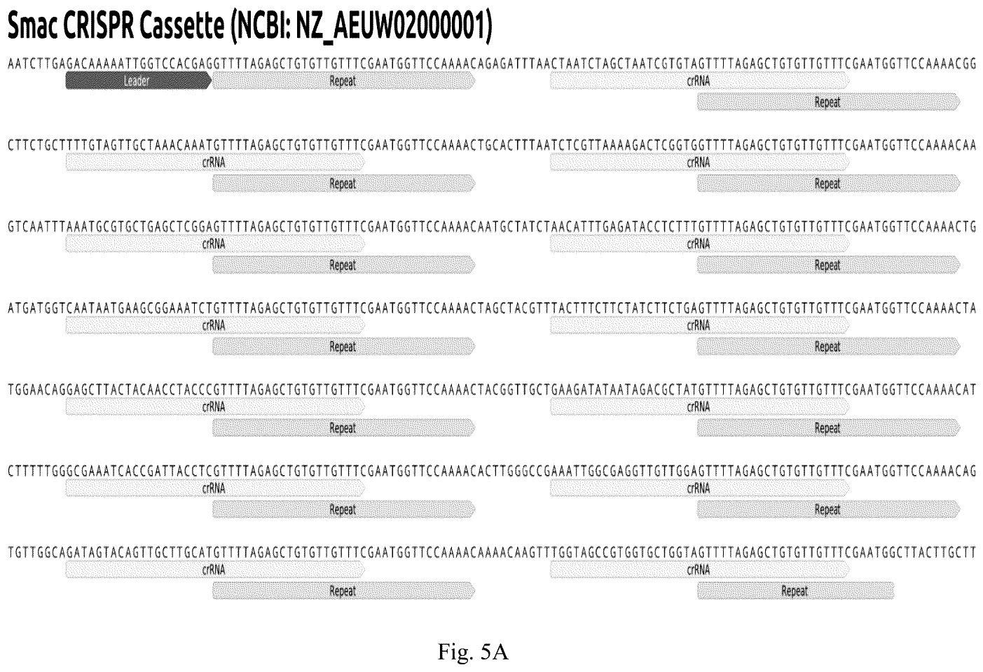

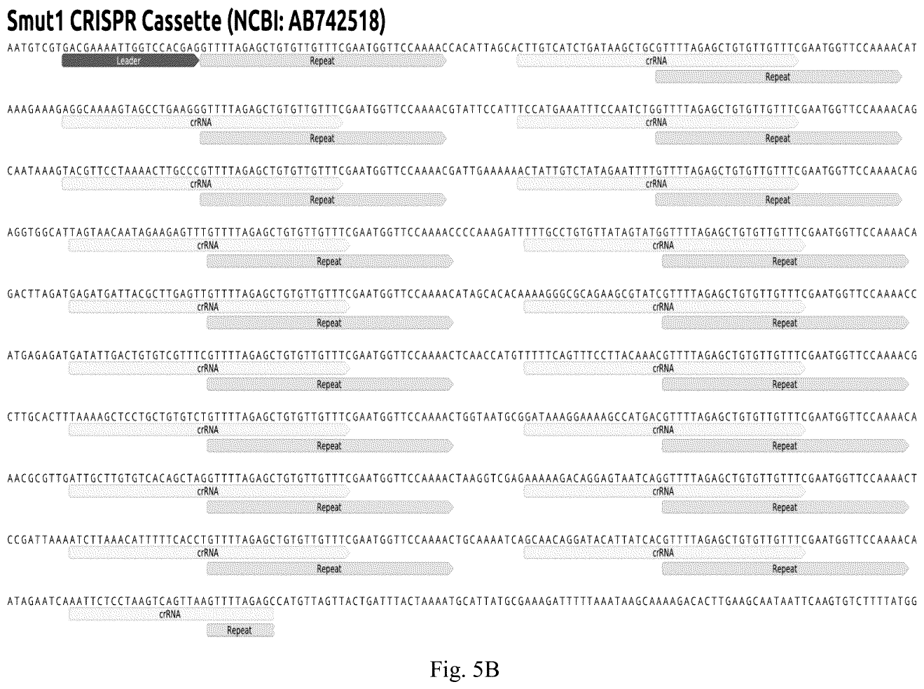

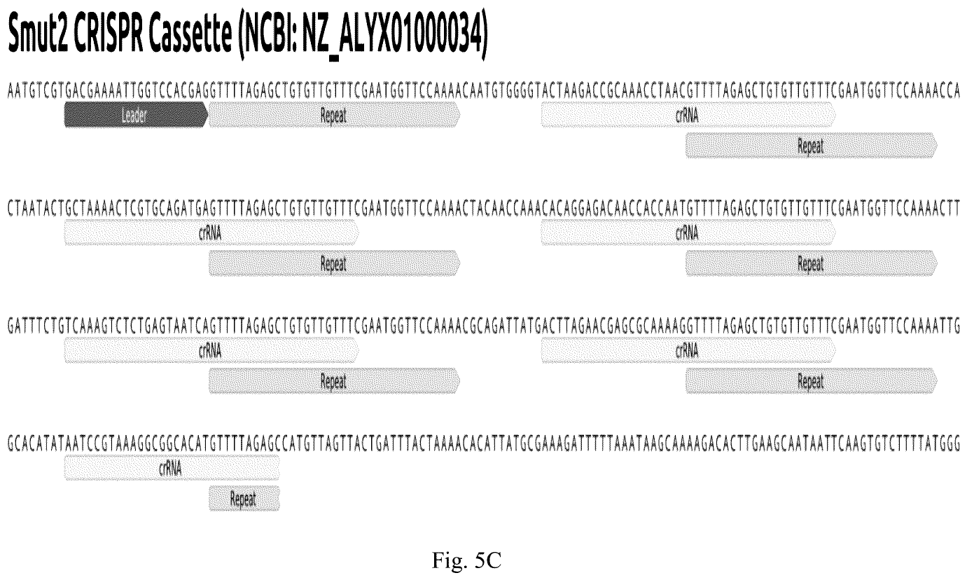

FIG. 5A-C depict annotated CRISPR cassettes obtained from the genomes corresponding to Smac (FIG. 5A), Smut1 (FIG. 5B), and Smut2 (FIG. 5C) orthologs that substitute both PAM-contacting arginine residues to glutamine.

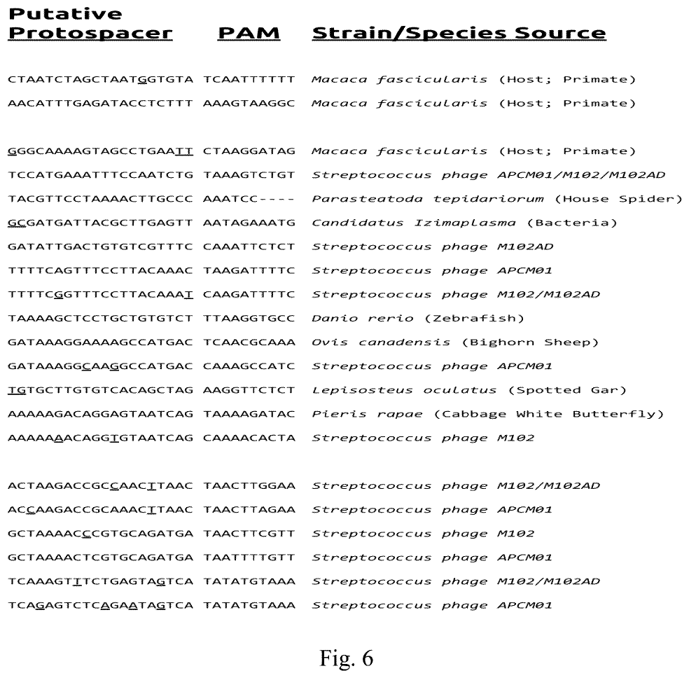

FIG. 6 depicts mappings of CRISPR cassette spacers to their putative target source for listed crRNA, identified via an online BLAST and/or SPAMALOT. SPAMALOT uncovered most cases of mismatch-tolerated mappings to Streptococcus phage. Underlined bases indicate mismatches that are tolerated for the mapping. Additional line spacing separates analysis for each CRISPR cassette.

To validate the predicted minimal A-rich PAM sequence of the described variants, a bacterial assay based upon lacI promoter repression of GFP expression, employing a fully randomized 8-nucleotide library of PAM sequences upstream of lacI, was utilized [Leenay, R.T. et al., “Identifying and visualizing functional PAM diversity across CRISPR-Cas systems”, Mol. Cell 62, 137-147 (2016)]. The library-containing plasmids were co-electroporated with a gRNA plasmid and a nuclease-activity deficient Spy-Mac Cas9 (dSpy-Mac Cas9) plasmid, all expressing different antibiotic resistance cassettes (Kanamycin, Ampicillin, Chloramphenicol, respectively). Transformants were collected in 5 ml of triple antibiotic-containing Luria Broth (LB) media. Overnight cultures were diluted to an ABS600 of 0.01 and cultured to an OD600 of 0.2. Cultures were analyzed and sorted on a FACSAria machine (Becton Dickinson). Events were gated based on forward scatter and side scatter and fluorescence was measured in the FITC channel (488 nm laser for excitation, 530/30 filter for detection), with at least 30,000 gated events for data analysis. Sorted GFP-positive cells were grown to sufficient density, and plasmids from the pre-sorted and sorted populations were then isolated, and the region flanking the nucleotide library was PCR amplified and submitted for Sanger sequencing (Genewiz).

The PAM preferences of several Streptococcus orthologs that change one or both of the critical PAM-contacts were experimentally assayed. Based on demonstrated examples of the PAM-interaction (PI) domain and guide RNA (gRNA) having cross-compatibility between Cas9 orthologs that are closely related and active, new variants were constructed by rationally exchanging the PI region of catalytically-“dead” Spy Cas9 (Spy dCas9) with those of the selected orthologs [Nishimasu, H. et al., “Crystal structure of Cas9 in complex with guide RNA and target DNA”, Cell 156, 935-949 (2014); Briner, A. E. et al., “Guide RNA functional modules direct Cas9 activity and orthogonality”, Molecular cell 56, 333-339 (2014)].

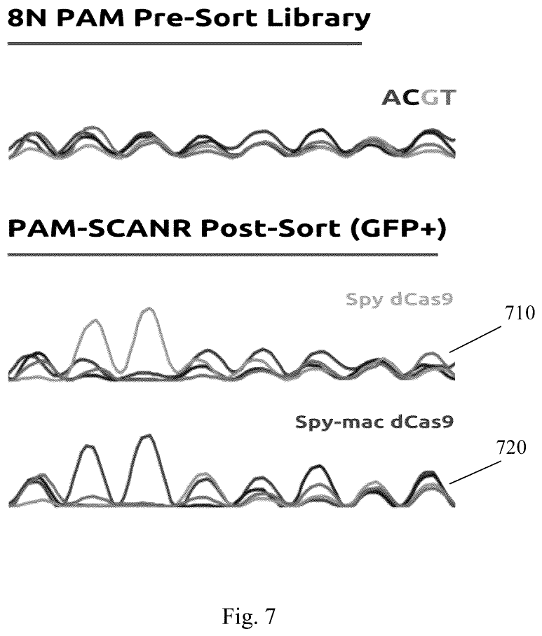

FIG. 7 depicts chromatograms representing the PAM-SCANR based enrichment of variant-recognizing PAM sequences from a 5′-NNNNNNNN-3′ library for Spy dCas9 710 and Spy-mac dCas9 720. The sequencing chromatograms demonstrate enrichment of A at positions 2and 3 in the PAM sequence for Spy-mac dCas9, as compared to the canonical 5′-NGG-3′or Spy Cas9. To further analyze base preferences at other positions in the PAM sequence, a randomized 6-nucleotide library, with A being held constant at positions 2 and 3, was utilized. The chromatograms of enriched sequences confirm the lack of specificity at downstream PAM positions, confirming the 5′-NAA-3′ specificity of Spy-Mac Cas9 in bacterial cells.

Assembled variants, including Spy-mac dCas9, were separately co-transformed into E. coli cells, along with guide RNA derived from S. pyogenes and an 8-mer PAM library of uniform base representation in the PAM-SCANR genetic circuit, established by others [Leenay, R. T. et al., “Identifying and visualizing functional PAM diversity across CRISPR-Cas systems”, Molecular Cell 62, 137-147 (2016)]. The circuit usefully up-regulates a green fluorescent protein (GFP) reporter in proportion to PAM-binding strength. Therefore, the GFP-positive cell populations were collected by flow cytometry and Sanger sequenced around the site of the PAM to determine position-wise base preferences in a corresponding variant's PAM recognition. Spy-mac dCas9, more so than Spy-mut dCas9, generated a trace profile that was most consistent with guanine-independent PAM recognition, along with a dominant specificity for adenine dinucleotides (FIG. 4).

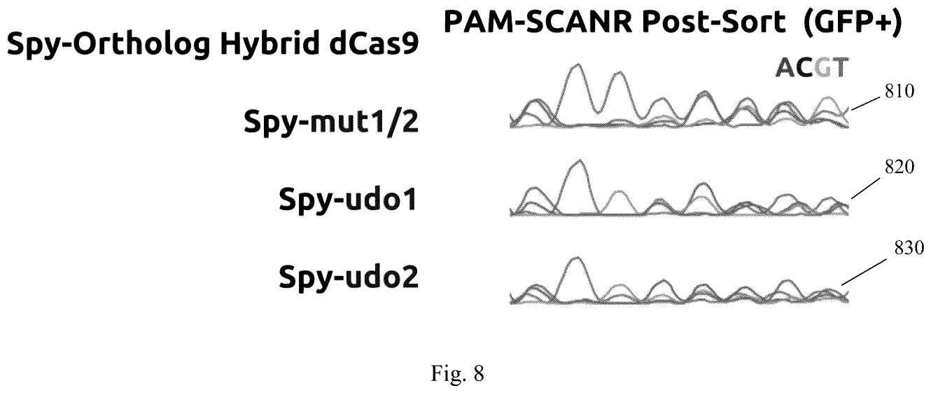

FIG. 8 depicts chromatograms representing the PAM-SCANR based enrichment of variant-recognizing PAM sequences from a 5′-NNNNNNNN-3′ library for Spy-Ortholog Hybrid dCas9. Shown in FIG. 8 are Spy-mut1/2 810, Spy-udo1 820, and Spy-udo2 830.

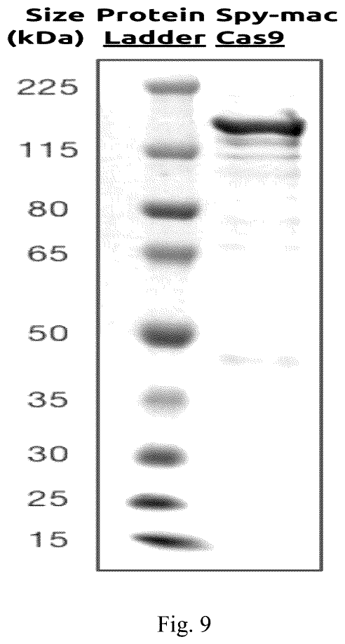

Nuclease-active enzymes were purified to continue probing the DNA target recognition potential and uniqueness of Spy-mac Cas9 [Anders, C. & Jinek, M., “In vitro enzymology of Cas9”, Methods in enzymology 546, 1-20 (2014); Lin, S., Staahl, B. T., Alla, R. K. & Doudna, J. A., “Enhanced homology-directed human genome engineering by controlled timing of CRISPR/Cas9 delivery”, eLife 3, e04766 (2014)]. FIG. 9 is an SDS-PAGE gel image of Spy-mac Cas9 after purification by affinity chromatography.

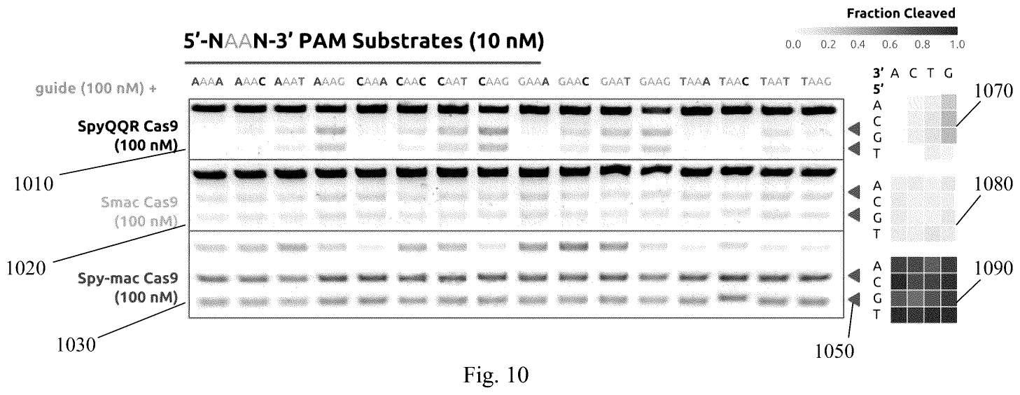

The ribonucleoprotein complex enzymes (composed of Cas9+crRNA+tracrRNA) were individually incubated with double-stranded target substrates of all 5′/3′-neighboring base combinations at an adenine dinucleotide PAM (5′-NAAN-3′). FIG. 10 depicts SYBR-stained agarose gels showing in vitro digestion of 10 nM 5′-NAAN-3′ substrates upon 16 minutes of incubation with 100 nM of purified ribonucleoprotein enzyme assemblies for SpyQQR Cas9 1010, Smac Cas9 1020, and Spy-mac Cas9 1030. Arrows 1050 distinguish banding of the cleaved products from uncleaved substrate (top band). Matrix plots 1070, 1080, 1090 summarize cleaved fraction calculations, which were carried out in a custom script for processing gel images.

Table 2 lists the sequence information for the in vitro digest reactions.

TABLE 2

| Name | Sequence |
|---|---|
| crRNA | rCrGrArArArGrGrUrUrUrUrGrCrArCrUrCrGrArC . . . |
| | rGrUrUrUrUrArGrArGrCrUrArUrGrCrU [SEQ ID No. 1] |
| tracrRNA (Spy) | rArGrCrArUrArGrCrArArGrUrUrArArArArU . . . |
| | rArArGrGrCrUrArGrUrCrCrGrUrUrArUrCrA . . . |
| | rArCrUrUrGrArArArArArGrUrG . . . |
| | rGrCrArCrCrGrArGrUrCrGrGrUrGrCrUrU [SEQ ID No. 2] |
| PAM Target | CGAAAGGTTTTGCACTCGACNNNNACCAACGAAAGGGCC |
| 5′-NNNN-3′ | [SEQ ID No. 3] |

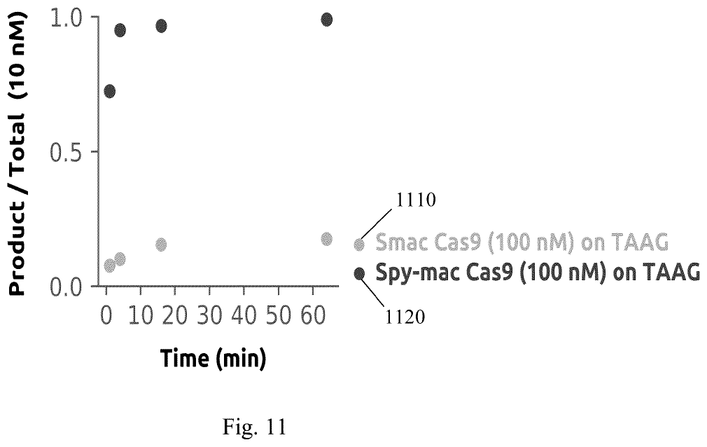

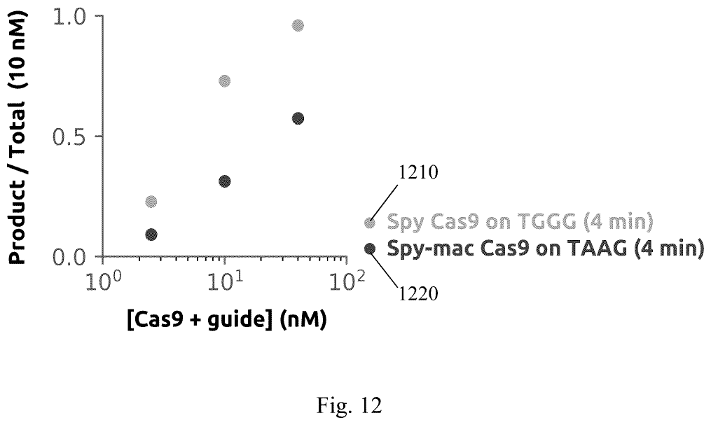

Brief 16-minute digestion indicated both wild-type Smac Cas9 and the hybrid Spy-mac Cas9 cleaved adjacently to 5′-NAAN-3′ motifs more broadly and evenly than the previously reported QQR variant. Spy-mac Cas9 distinguished itself further with rapid DNA-cutting rates that resemble the fast digest kinetics of Spy Cas9 [Gong, S., Yu, H. H., Johnson, K. A. & Taylor, D. W., “DNA unwinding is the primary determinant of CRISPR-Cas9 activity”, Cell Reports 22, 359-371 (2018)]. FIG. 11 is a plot of timecourse measurements of target DNA substrate cleavage for Smac Cas9 1110 and Spy-mac Cas9 1120. FIG. 12 is a plot of DNA substrate cleavage plotted as a function of 0.25:1, 1:1, and 4:1 molar ratios of ribonucleoprotein to target for wild-type Spy Cas9 1210 and hybrid Spy-mac Cas9 1220.

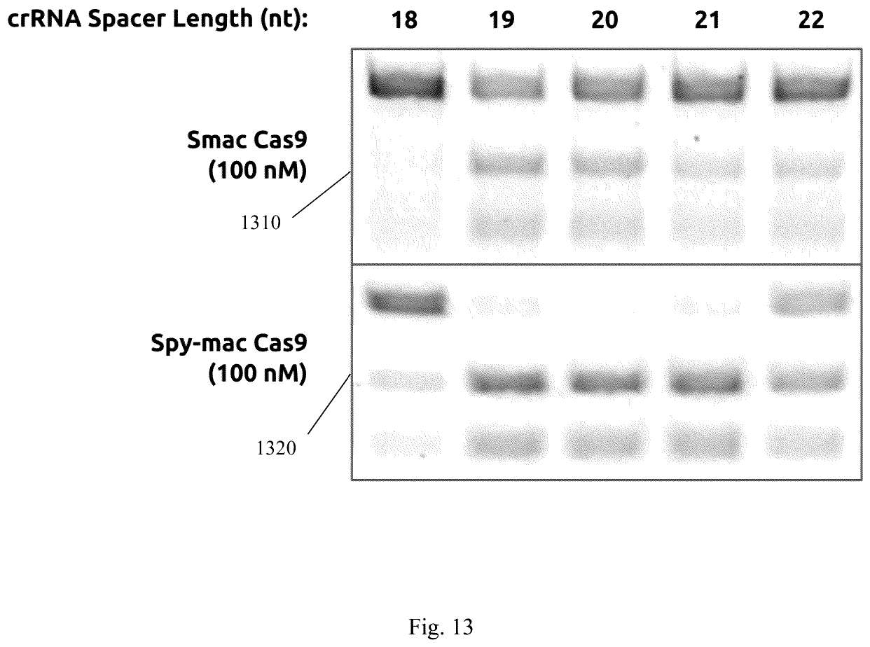

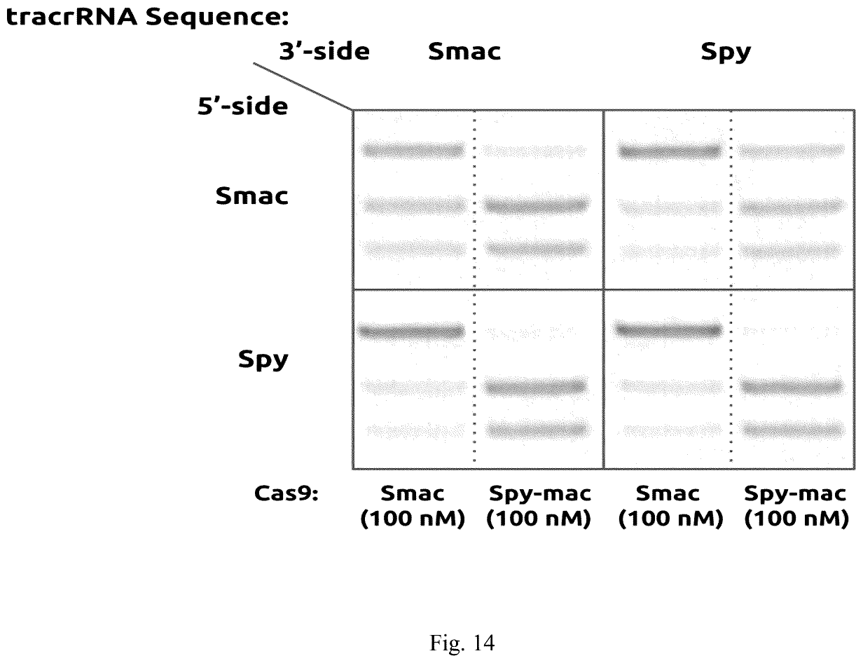

Reactions that used varying crRNA spacer lengths and tracrRNA sequence were run, as the latter differs slightly between the S. macacae and S. pyogenes genomes. In FIG. 13, SYBR-stained agarose gels running in vitro digestion reactions are shown that assay dependencies on crRNA spacer length for Smac Cas9 1310 and Spy-mac Cas9 1320. In FIG. 14, SYBR-stained agarose gels running in vitro digestion reactions are shown that assay dependencies on tracrRNA sequence origin. The results in FIGS. 13 and 14 were produced by Digest (16 min) of TAAG PAM substrate (10 nM) with cRNA and tracrRNA (100 nM).

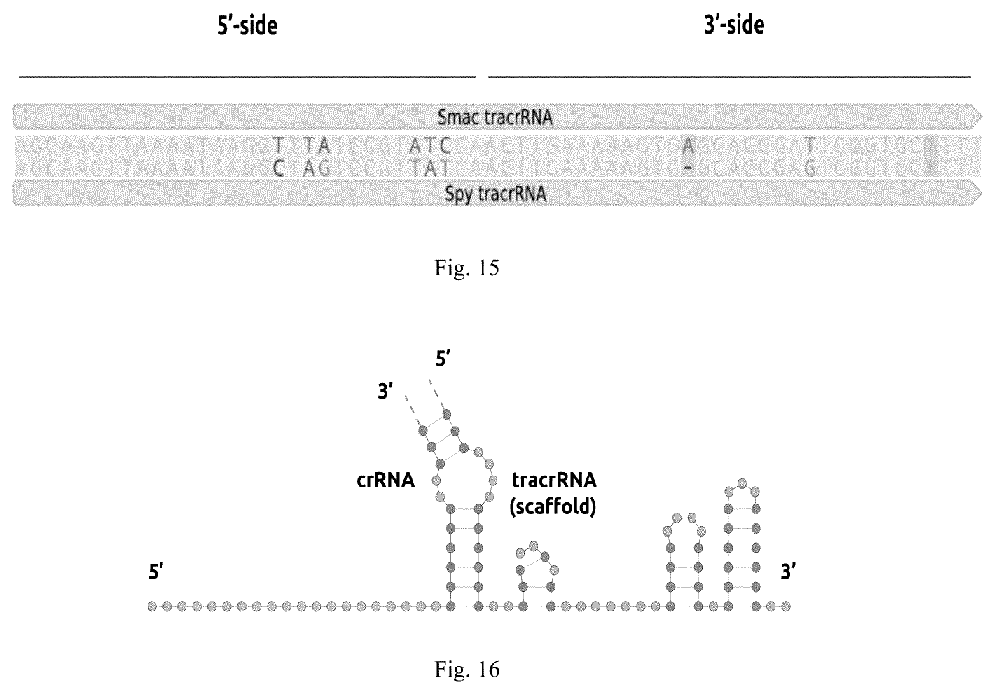

FIGS. 15 and 16 depict sequence alignment (Genewiz software) (FIG. 15) of tracrRNA from S. pyogenes and S. mutans highlighted in a color code that reflects the base-pairing in their duplex gRNA secondary structure (FIG. 16). Neither of these two parameters compensated for the slower cleavage rate of Smac Cas9, but marginal improvement was seen in the activity of the wild-type form with its native tracrRNA, which comports with the interface of the guide-Cas9 interaction being mostly outside of the PI domain.

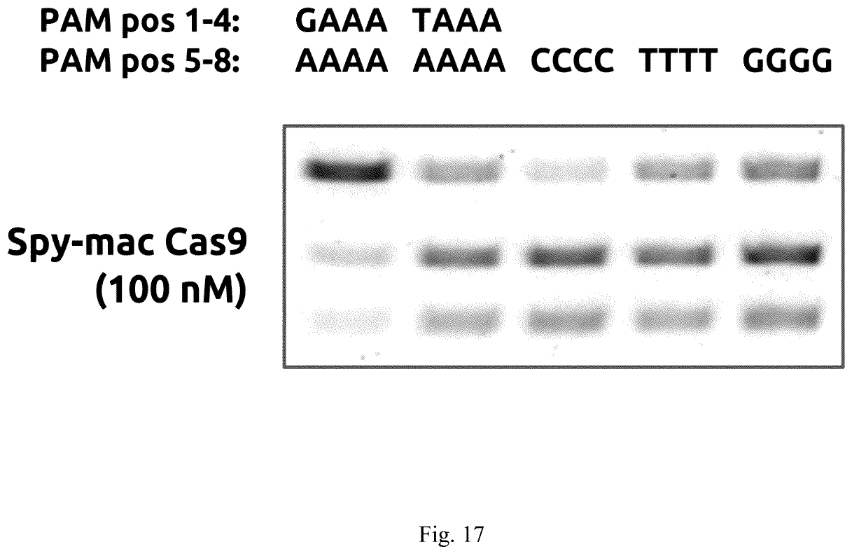

To crucially verify that an adenine dsDNA dinucleotide is sufficient for Cas9 PAM recognition, it was confirmed that Spy-mac Cas9 remains active on targets that set the next four downstream bases to the same nucleotide (e.g. 5′-TAAGXXXX-3′, for X all fixed to A, C, G, or T). In FIG. 17, SYBR-stained agarose gels running in vitro digestion reactions are shown that assay dependencies on positions 5-8 in the PAM sequence. The results in FIG. 17 were produced by Digest (16 min) of TAAG PAM substrate (10 nM) with cRNA and tracrRNA (100 nM).

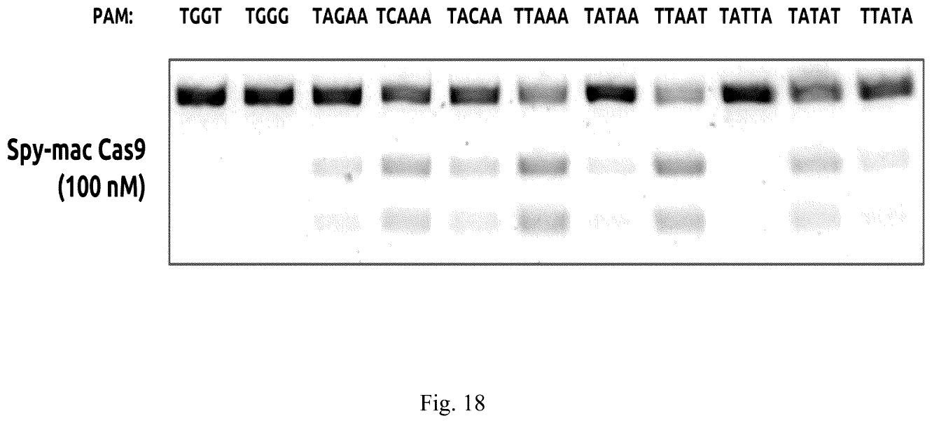

Additionally, a moderate yield of cleaved products on examples of 5′-NBBAA-3′, 5′-NABAB-3′, 5′-NBABA-3′ PAM sequences (where B is the IUPAC symbol for C, G, or T) were observed, revealing an even broader tolerance for increments to the dinucleotide position or adenine adjacency. In FIG. 18, SYBR-stained agarose gels running in vitro digestion reactions are shown that assay dependencies on increments to the distribution of adenine content in positions 1-5 in the PAM sequence. The results in FIG. 18 were produced by Digest (16 min) of TAAG PAM substrate (10 nM) with cRNA and tracrRNA (100 nM).

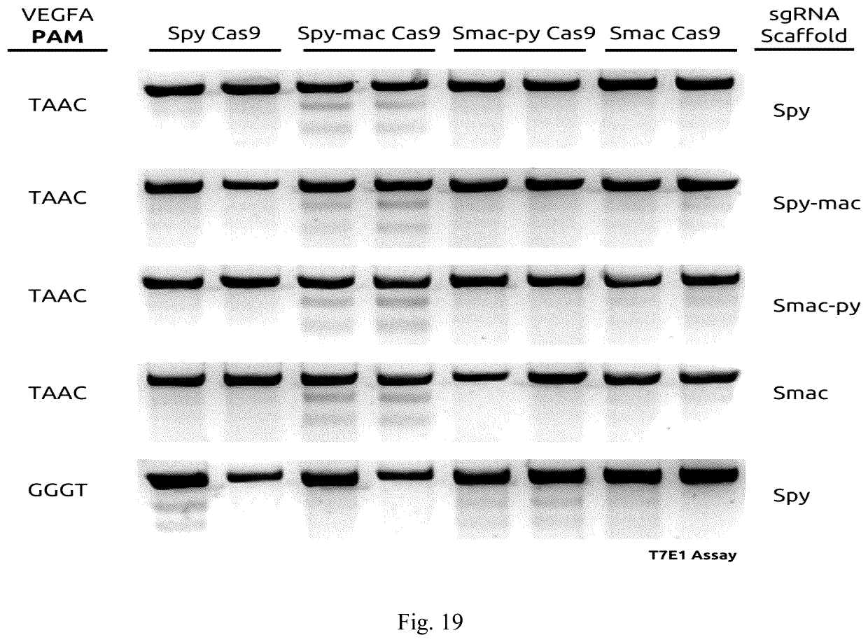

The capacity for gene modification in human cells of Spy-mac Cas9 was also investigated. FIG. 19 depicts detection of genomic modification in SYBR-stained agarose gels running T7EI digests upon targeting a single PAM site with combinations of wild-type plus hybrid variants of Cas9 and guide scaffold (tracrRNA sequence) from S. pyogenes and S. macacae.

A human embryonic kidney (HEK293T) cell line was transfected with plasmids that encode Smac Cas9 or Spy-mac Cas9, and co-expressed single-guide RNA molecules that target the VEGFA gene locus at sites representing a breadth of 5′-NAAN-3′ PAM diversity. Table 3 lists sequence information for genome editing in human cells.

TABLE 3

| Name | Sequence |
|---|---|
| sgRNA for Cas9 | N20(Target) GTTTTAGAGCTATGCTG . . . |
| | GAAACAGCATAGCAAGTTAAAAT . . . |
| | AAGGCTAGTCCGTTATCAACTTGAAA . . . |
| | AAGTGGCACCGAGTCGGTGCTT polyT |
| | [SEQ ID No. 4] |
| gRNA for AsCas12a | TAATTTCTACTCTTGTAGAT N20(Target) polyT |
| | [SEQ ID No. 5] |
| gRNA for LbCas12a | AATTTCTACTAAGTGTAGAT N20(Target) polyT |
| | [SEQ ID No. 6] |
| Target for CAAATTCC PAM w/ Cas9 | GAACCCGGATCAATGAATAT [SEQ ID No. 7] |
| Target for CAAATTCC PAM w/ Cas12a | ATATTCATTGATCCGGGTTC [SEQ ID No. 8] |
| Target for CAACCCCA PAM w/ Cas9 | GCTCCCCGCTCCAACACCCT [SEQ ID No. 9] |
| Target for CAACCCCA PAM w/ Cas12a | AGGGTGTTGGAGCGGGGAGC [SEQ ID No. 10] |
| Target for CAAGCCGT PAM w/ Cas9 | GGGAAGTAGAGCAATCTCCC [SEQ ID No. 11] |
| Target for CAAGCCGT PAM w/ Cas12a | GGGAGATTGCTCTACTTCCC [SEQ ID No. 12] |
| Target for CAATGTGC PAM w/ Cas9 | GCCACAGTGTGTCCCTCTGA [SEQ ID No. 13] |
| Target for CAATGTGC PAM w/ Cas12a | TCAGAGGGACACACTGTGGC [SEQ ID No. 14] |
| Target for TAACCTCA PAM w/ Cas9 | GCTCAGGCCCTGTCCGCACG [SEQ ID No. 15] |
| Target for TAACCTCA PAM w/ Cas12a | CGTGCGGACAGGGCCTGAGC [SEQ ID No. 16] |
| Target for TAAGGCCC PAM w/ Cas9 | GTTCCATCGGTATGGTGTCC [SEQ ID No. 17] |
| Target for TAAGGCCC PAM w/ Cas12a | GGACACCATACCGATGGAAC [SEQ ID No. 18] |
| Target for GAAGTCGA PAM w/ Cas9 | GGTAGCAAGAGCTCCAGAGA [SEQ ID No. 19] |
| Target for GAAGTCGA PAM w/ Cas12a | TCTCTGGAGCTCTTGCTACC [SEQ ID No. 20] |
| Target for GAAAGTGA PAM w/ Cas9 | GATTGGCGAGGAGGGAGCAG [SEQ ID No. 21] |
| Target for GAAAGTGA PAM w/ Cas12a | CTGCTCCCTCCTCGCCAATC [SEQ ID No. 22] |
| Target for GAAACCAG PAM w/ Cas9 | GCCTGGAAATAGCCAGGTCA [SEQ ID No. 23] |
| Target for GAAACCAG PAM w/ Cas12a | TGACCTGGCTATTTCCAGGC [SEQ ID No. 24] |
| Target for AAACCAGC PAM w/ Cas9 | GCTGGAAATAGCCAGGTCAG [SEQ ID No. 25] |
| Target for AAACCAGC PAM w/ Cas12a | CTGACCTGGCTATTTCCAGC [SEQ ID No. 26] |
| Target for AAAGTGAG PAM w/ Cas9 | GTTGGCGAGGAGGGAGCAGG [SEQ ID No. 27] |
| Target for AAAGTGAG PAM w/ Cas12a | CCTGCTCCCTCCTCGCCAAC [SEQ ID No. 28] |
| Target for AAATTCCA PAM w/ Cas9 | GACCCGGATCAATGAATATC [SEQ ID No. 29] |
| Target for AAATTCCA PAM w/ Cas12a | GATATTCATTGATCCGGGTC [SEQ ID No. 30] |

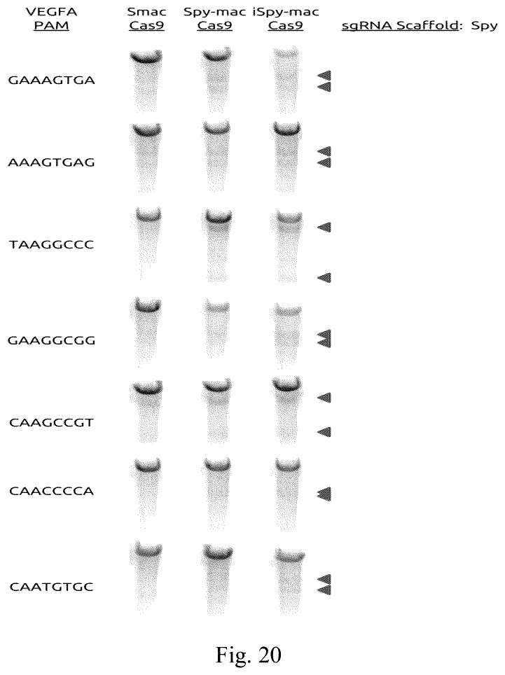

Consistent with in vitro observations, it was found that Spy-mac Cas9 was more efficient than Smac Cas9 at mediating enzymatically-detected (T7 EndonucleaseI) genomic insertion/deletion (indel) mutations. Spymac Cas9 also proved capable of generating indels with variable efficiency on instances of any directly 5′- or 3-neighboring base for 5′-NAAG-3′ or 5′-CAAN-3′ PAM sequences. FIG. 20 depicts a diversity of PAM sequences with the wild-type and engineered variants that include the Smac Cas9 PI domain. Arrows point to the banding from products digested by T7EI, which is used to estimate gene modification efficiencies.

To address sites with low modification rates, two mutations (R221K and N394K) were introduced into Spy-mac Cas9 that can raise gene knock-out percentages and had been previously identified by deep mutational scans of Spy Cas9 [Spencer, J. M. & Zhang, X., “Deep mutational scanning of S. pyogenes Cas9 reveals important functional domains”, Scientific Reports 7 (2017)]. This variant is referred to as an “increased” editing Spy-mac Cas9 (iSpy-mac Cas9), due to its similarly elevated modification rates on most targets.

The gene editing performance of the nucleases derived from Streptococcus macacae Cas9 was benchmarked against orthologs of Cas12a by making use of their common AT-rich PAM specificity (FIGS. 5A-C) [Yamano, T. et al., “Structural basis for the canonical and non-canonical PAM recognition by CRISPR-Cpf1”, Molecular Cell 67, 633-645.e3 (2017); Gao, L. et al., “Engineered Cpf1 variants with altered PAM specificities”, Nature biotechnology 35, 789-792 (2017)]. Cas12a orthologs known for efficient gene editing from Acidaminococcus sp. BV3L6 (AsCas12) and Lachnospiraceae bacterium ND2006 (LbCas12) were included [Kim, D. et al., “Genome-wide analysis reveals specificities of Cpf1 endonucleases in human cells”, Nature biotechnology 34, 863-868 (2016)]. The selection of target sites permits overlapping PAM recognition between these Cas9 and Cas12a nucleases by guiding the Cas12a variants with the reverse complemented spacer sequences of those guiding Cas9 variants.

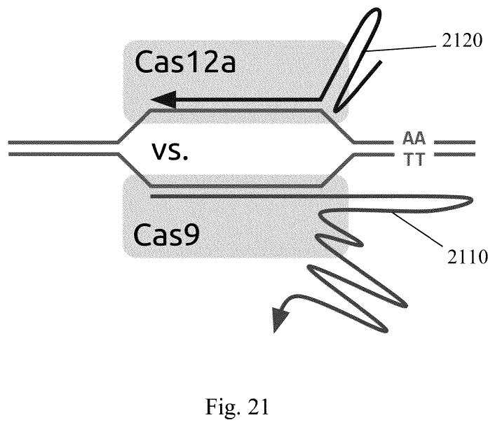

FIG. 21 is a schematic diagram for matching Cas9 2110 and Cas12a 2120 guides in a manner that enforces their recognition of the same PAM sequence and therefore facilities their comparison (a “Cas12a vs Cas9 Comparator”). The Cas9 and Cas12a thereby targeted opposite strands, yet were constrained to recognize the same PAM site and preserve important features for guide RNA effectiveness (e.g. distribution of purines/pyrimidines, directionality of target-matching in relation to the PAM, and GC-content) [Thyme, S. B., Akhmetova, L., Montague, T. G., Valen, E. & Schier, A. F., “Internal guide RNA interactions interfere with Cas9-mediated cleavage”, Nature communications 7, 11750 (2016); Labuhn, M. et al., “Refined sgRNA efficacy prediction improves large- and small-scale CRISPR-Cas9 applications”, Nucleic acids research 46, 1375-1385 (2018)].

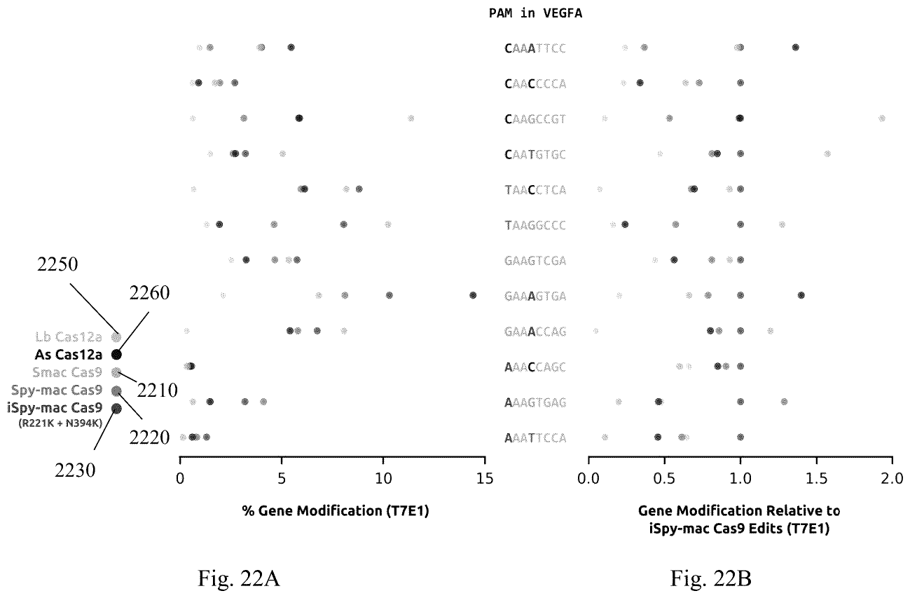

FIGS. 22A-B are dot plots of absolute (FIG. 22A) and relative (FIG. 22B) gene modification efficiency in HEK293T cells by Cas9 (Smac Cas9 2210, Spy-mac Cas9 2220, iSpy-mac Cas9 2230) and Cas12a (Lb Cas12a 2250, As Cas12a 2260) variants targeting common PAM sequences located in the VEGFA gene. Values were quantified in a T7EI-based assay and are consistent with biological duplicates that were run in parallel.

Cas12a and Cas9 activity was compared explicitly on an endogenous genomic locus. For each site examined, iSpy-mac Cas9 consistently generated a larger indel percentage than either AsCas12a or LbCas12a—never exhibiting less activity than the lower-editing of the two Cas12 proteins—if not generating the largest overall percentage.

A window of four nucleotides in the VEGFA locus was selected in a sequence context such that any other reported CRISPR endonuclease capable of gene modification would not allow their base editing with a cytidine deaminase-fused enzyme [Mir, A., Edraki, A., Lee, J. & Sontheimer, E. J., “Type II-c CRISPR-cas9 biology, mechanism, and application”, ACS Chemical Biology 13, 357-365 (2017)]. A Spy-mac Cas9 base editor has a distinct targeting range to implementations that use Cas12a, since current base editing methods directly modify the non-target strand and, in order to recognize the same PAM site, the two enzyme types must target in opposite orientations [Yamano, T. et al., “Crystal structure of Cpf1 in complex with guide RNA and target DNA”, Cell 165, 949-962 (2016); Li, X. et al., “Base editing with a Cpf1-cytidine deaminase fusion”, Nature Biotechnology 36, 324-327 (2018)]. Hence, Cas9 base editing architectures utilize their ability to nick on the guide-pairing target side of the R-loop structure (ribonucleoprotein bound and matched to DNA) to transfer a base edit in a manner that templates from the modified nontarget strand [Gaudelli, N. M. et al., “Programmable base editing of a-t to g-c in genomic DNA without DNA cleavage”, Nature 551, 464-471 (2017)].

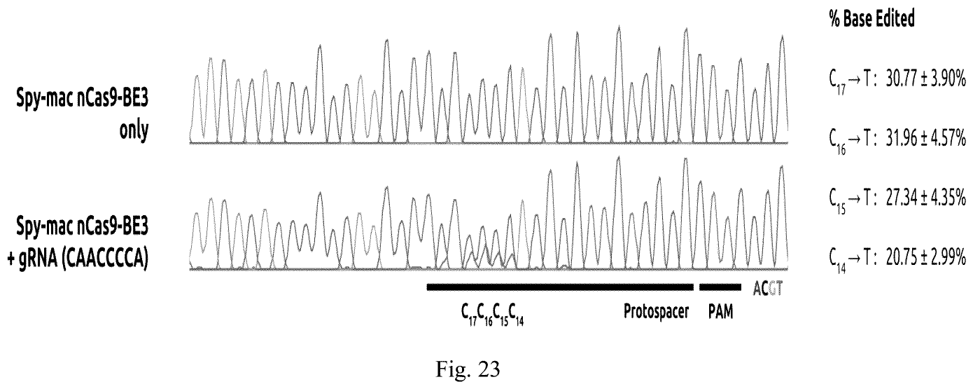

Accordingly, HEK293T cells were co-transfected with a nickase form of Spy-mac Cas9 derived from the previously reported BE3 architecture for cytosine base editing (Spy-mac nCas9-BE3) and the gRNA plasmid targeting a PAM downstream of the selected nucleotides [Komor, A. C., Kim, Y. B., Packer, M. S., Zuris, J. A. & Liu, D. R., “Programmable editing of a target base in genomic DNA without double-stranded DNA cleavage”, Nature 533, 420-424 (2016)]. Robust levels of base editing in harvested cells were measured, which exhibited 20% to 30% cytosine to thymine conversion at these positions. FIG. 23 is a chromatogram depicting a genomic base editing demonstration for the targeted conversion of cytosines to thymines with Spy-mac nCas9-BE3. Analysis on the efficiency was carried out in a custom Sanger sequencing trace file processing script called BEEP.

Despite previous reports indicating base editing rates are generally lower than gene modification rates for the same target, a significant gain was observed compared to the indel formation when using double-strand breaking enzymes for this PAM site [Hu, J. H. et al., “Evolved Cas9 variants with broad PAM compatibility and high DNA specificity”, Nature 556, 57-63 (2018)]. Such discrepancy is likely explained by scaling to more sites for larger gene modification experiments, and possibly by differing codon usage outside of the PI domain. Recent work shows that higher editing rates can be achieved by optimizing such codon selection, nuclear-localization sequences/linkers, protein solubility, delivery methods, and sortable labeling of transfected cells [Koblan, L. W. et al., “Improving cytidine and adenine base editors by expression optimization and ancestral reconstruction”, Nature biotechnology 36, 843-846 (2018); Wang, T., Badran, A. H., Huang, T. P. & Liu, D. R., “Continuous directed evolution of proteins with improved soluble expression”, Nature chemical biology (2018); Liang, X. et al., “Rapid and highly efficient mammalian cell engineering via Cas9 protein transfection”, Journal of biotechnology 208, 44-53 (2015); Duda, K. et al., “High-efficiency genome editing via 2a-coupled co-expression of fluorescent proteins and zinc finger nucleases or CRISPR/Cas9 nickase pairs”, Nucleic acids research 42, e84 (2014)].

The invention therefore includes the application of Smac Cas9 and Spy-Mac Cas9 as tools for genome engineering in human cells. Briefly, the coding sequence of the described Cas9 variants are transiently transfected, using standard lipofection reagents (e.g.

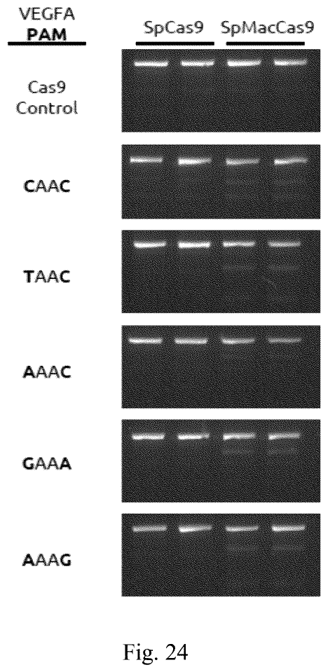

Lipofectamine 2000), as plasmids under the control of an Elongation Factor 1-alpha (EF1-a) promoter in HEK293T cells along with guide RNA vectors under the control of a U6 promoter containing spacer sequences targeting various 5′-NAA-3′ PAM sequences at the standard VEGFA locus. After 5 days post transfection, individual cells are harvested for genomic extraction to allow for an approximately one kilobase (kb) window around the target to be amplified via polymerase chain reaction (PCR). T7E1 indel analysis demonstrates effective cleavage on 5′-NAA-3′ targets with a variety of base combinations at positions 1 and 4 in the PAM sequence, as shown in FIG. 24. Indel formation can be further verified on Sanger sequencing results utilizing the TIDE algorithm or ICE (Synthego). The invention further includes utilizing the described variants for applications such as, but not limited to, specific base conversions and gene regulation applications, such as transcriptional activation and repression.

For in vitro and in vivo applications, the invention is compatible with additional delivery methods used for other CRISPR-Cas9 systems including, but not limited to, electroporation, viral infection, and nanoparticle injection. Embodiments can co-deliver the invention as a coding nucleic acid or protein, along with a gRNA. Components can also be stably expressed in cells.

At least the following aspects, implementations, modifications, and applications of the described technology are contemplated by the inventors:
(1) An isolated Streptococcus macacae Cas9 (ScCas9) protein or transgene expression thereof.
(2) Naturally-occurring and engineered CRISPR-associated DNA endonucleases with PAM interacting domain (PID) amino acid sequences that are at least 80% identical to that of the isolated protein in (1).
(3) CRISPR-associated DNA endonucleases with a PAM specificity of “NAA” or “NA”.
(4) An isolated, engineered Streptococcus pyogenes Cas9 (Spy Cas9) protein with its PID as either the PID amino acid composition of the isolated protein in (1) or that of those in (2).
(5) The method of altering expression of at least one gene product comprising introducing into a eukaryotic cell containing and expressing a DNA molecule having a target sequence and encoding the gene product an engineered, non-naturally occurring Clustered Regularly Interspaced Short Palindromic Repeats (CRISPR)-CRISPR associated (Cas) (CRISPR-Cas) system comprising one or more vectors comprising: (a) a regulatory element operable in a eukaryotic cell operably linked to at least one nucleotide sequence encoding a CRISPR system guide RNA that hybridizes with the target sequence, and (b) a second regulatory element operable in a eukaryotic cell operably linked to a nucleotide sequence encoding one or more of the proteins in (1)-(4), wherein components (a) and (b) are located on same or different vectors of the system, whereby the guide RNA targets the target sequence and one or more of the proteins in (1)-(4) cleave the DNA molecule, whereby expression of the at least one gene product is altered, and wherein the protein(s) and the guide RNA do not naturally occur together.

### Materials and Methods

Selection of Streptococcus Cas9 Orthologs of Interest. All Cas9 orthologs from the Streptococcus genus were downloaded from the online UniProt database. These were then downselected by pair-wise alignment to Spy Cas9 using a Blosum62 cost matrix in the Genewiz software package and discarding orthologs with less than 70% agreement with the Spy Cas9 sequence. The remaining 115 orthologs were used to generate a sequence logo (Weblogo), and were manually selected for divergence at positions aligned to residues critical for the PAM interaction of Spy Cas9. The SPAMALOT pipeline was implemented as previously reported [Chatterjee, P., Jakimo, N. & Jacobson, J. M., “Divergent pam specificity of a highly-similar spcas9 ortholog”, bioRxiv (2018)].

PAM-SCANR Bacterial Fluorescence Assay. Sequences encoding the PAM-interaction domains of selected Cas9 orthologs were synthesized as gBlock fragments by Integrated DNA Technologies (IDT) and inserted via a New England Biolabs (NEB) Gibson Assembly reaction into the C-terminus of a low-copy plasmid containing Spy dCas9 (Beisel Lab, NCSU). The hybrid protein constructs were transformed into electrocompetent E. coli cells with additional PAM-SCANR components as previously established [Leenay, R. T. et al., “Identifying and visualizing functional PAM diversity across CRISPR-cas systems”, Molecular Cell 62, 137-147 (2016)]. Overnight cultures were analyzed and sorted on a Becton Dickinson (BD) FACSAria machine. Sorted GFP-positive cells were grown to sufficient density, and plasmids from the pre-sorted and sorted populations were then isolated. The region flanking the nucleotide library was PCR amplified and submitted for Sanger sequencing (Genewiz). The choromatograms from received trace files were inspected for post-sorted sequence enrichments relative to the pre-sorted library.

Purification of and DNA cleavage with Selected Nucleases. The gBlock (IDT) encoding the PAM-interaction domain of S. macacae was inserted into a bacterial protein expression/purification vector containing wild-type S. pyogenes Cas9 fused to the His6-MBP-tobacco etch virus (TEV) protease cleavage site at the N-terminus (pMJ915, Addgene plasmid #69090). The resulting hybrid Spy-mac Cas9 protein expression construct was sequence-verified by a next-generation complete plasmid sequencing service (CCBI DNA Core Facility at Massachusetts General Hospital). The hybrid-protein construct was then transformed into BL21 Rosetta 2TM (DE3) (MilliporeSigma), and a single colony was picked for protein expression and inoculated in 2xYT and a final concentration of 1% glucose. An aliquot of overnight culture grown at 37 Celsius (5 ml) was used to re-inoculate in 1 L 2xYT and grown at 37 Celsius to a cell density of OD600 0.6, at which point the temperature was lowered to 18 Celsius and His-MBP-TEV-SpyMac Cas9 expression was induced by supplementing with 0.2 mM IPTG and grown for 18 hours before harvest.

Cells were then lysed with BugBuster™ Protein Extraction Reagent, supplemented with 1 mg/ml lysozyme solution (MilliporeSigma), 125 Units/gram cell paste of Benzonase™ Nuclease (MilliporeSigma), and complete, EDTA-free protease inhibitors (Roche Diagnostics Corporation). The lysate was clarified by centrifugation, including a final spin with a pre-chilled Steriflip™ 0.45 micron filter (MilliporeSigma). The clarified lysate was incubated with Ni-NTA resin (Qiagen) at 4 Celsius for 1 hour and subsequently applied to an Econo-Pac™ chromatography column (Bio-Rad Laboratories). The protein-bound resin was washed extensively with wash buffer (20 mM Tris pH 8.0, 800 mM KC1, 20 mM imidazole, 10% glycerol, 1 mM TCEP) and His-tagged Spy-mac protein was eluted in wash buffer (20 mM HEPES, pH 8.0, 500 mM KC1, 250 mM imidazole, 10% glycerol). ProTEV™ Plus protease (Promega, Madison) was added to the pooled fractions and dialyzed overnight into storage buffer (20 mM HEPES, pH 7.5, 500 mM KC1, 20% glycerol) at 4 Celsius using Slide-A-Lyzer™ dialysis cassettes with a molecular weight cut-off of 20 KDa (ThermoFisher Scientific). The sample was then incubated again with Ni-NTA resin for 1 hour at 4 Celsius with gentle rotation and applied to a chromatography column to remove the cleaved His tag. The protein was eluted with wash buffer (20 mM Tris pH 8.0, 800 mM KC1, 20 mM imidazole, 10% glycerol, 1 mM TCEP) and fractions containing cleaved protein were verified once more by SDS-PAGE and Coomassie staining, then pooled, buffer exchanged into storage buffer, and concentrated. The concentrated aliquots were measured based on their light-absorption (Implen Nanophotometer) and flash-frozen at −80 Celsius for storage or used directly for in vitro cleavage assays.

The crRNA and tracrRNA guide components were procured in the form of HPLC-purified RNA oligos (IDT) and resuspended in 1X IDTE pH 7.5 solution (IDT). Duplex crRNA-tracrRNA guides were annealed at 1 uM concentration in duplex buffer (IDT) by a protocol of rapid melting followed by gradual cooling. Target substrates were PCR amplified from assemblies of the PAMSCANR plasmid with a fixed PAM sequence. In vitro digestion reactions with 10 nM target and typically a 10-fold excess of enzyme components were prepared on ice and then incubated in a thermal cycler at 37 Celsius. Reactions were halted after at least 1 minute of incubation by subsequent heat denaturation at 65 Celsius for 5 minutes and run on a 2% TAE-agarose gel stained with DNA-intercalating SYBR dye (Invitrogen). Gel images were recorded from blue-light exposure and analyzed in a Python script. Cleavage fraction measurements were quantified by the relative intensity of substrate and product bands as:

$$\% cleaved fraction = \frac{integrated intensity of product bands}{integrated intensity of all}$$

Gene Modification Analysis and Software. The gBlock (IDT) encoding the PAM-interaction domain of S. macacae was swapped into the Spy Cas9 mammalian expression plasmid OG5209 (Oxford Genetics). Plasmids for Cas12a protein plus Cas9 and Cas12a guide construction were Addgene plasmid 78741, 78742, 78743, 78744. HEK293T cells were maintained in DMEM supplemented with 100 units/ml penicillin, 100 mg/ml streptomycin, and 10% fetal bovine serum (FBS). sgRNA plasmid (62.5 ng) and nuclease plasmid (187.5 ng) were transfected into cells as duplicates (5×104/well in a 96-well plate) with Lipofectamine 3000 (Invitrogen) in Opti-MEM (Gibco). After 5 days post-transfection, genomic DNA was extracted using QuickExtract Solution (Epicentre), and genomic loci were amplified by PCR utilizing the KAPA HiFi HotStart ReadyMix (Kapa Biosystems). For indel analysis, the T7EI reaction was conducted according to the manufacturer's instructions and equal volumes of products were analyzed on a 2% agarose gel stained with SYBR Safe (Thermo Fisher Scientific). Gel image files were analyzed with a Python script. Boundaries of cleaved and uncleaved bands of interest were hard-coded for each duplicate set of Cas variants with a common target, and the areas under the corresponding peaks were measured and calculated as the fraction cleaved of the total product. Percent gene modification was calculated as:

$$\% gene modification = 100 \times ( 1 - {( 1 - fraction cleaved )}^{1 / 2} )$$

Base Editing Analysis and Software. The gBlock (IDT) encoding the PAM-interaction domain of S. macacae was swapped into a mammalian expression plasmid for cytosine to thymine base editing (Addgene plasmid 73021). HEK293T (ATCC R CRL-3216™) cells (MilliporeSigma, Burlington, MA) were maintained in DMEM supplemented with 100 units/ml penicillin, 100 mg/ml streptomycin, and 10% fetal bovine serum (FBS). sgRNA (500 ng) and BE3 plasmids (500 ng) were transfected into cells as duplicates (2×105/well in a 24-well plate) with Lipofectamine 3000 (Invitrogen) in Opti-MEM (Gibco). After 5 days post-transfection, genomic DNA was extracted using QuickExtract Solution (Epicentre), and the VEGFA genomic locus was amplified by PCR utilizing the KAPA HiFi HotStart ReadyMix (Kapa Biosystems). Amplicons were purified and submitted for Sanger sequencing (Genewiz). For base conversion analysis, an automated Python script called BEEP, employing the pandas data manipulation library and BioPython package, was utilized to align base-calls of an input ab1 file to first determine the absolute position of the target within the file, and subsequently to measure the peak values for each base at the indicated position in the spacer. Finally, editing percentages of specified base conversions were calculated and normalized to that of an unedited control. Conversion efficiencies are reported as the average of two independent duplicate reactions±standard deviation.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. An isolated, engineered Streptococcus pyogenes Cas9 (SpyCas9) protein comprising SEQ ID No: 31 or SEQ ID No. 32, wherein said SpyCas9 is modified to have a Protospacer Adjacent Motif (PAM) Interacting Domain (PID) amino acid sequence with at least 80% identity to the amino acid composition of the PID of an isolated wild-type Streptococcus macacae Cas9 (Smac Cas9) protein comprising SEQ ID No: 33, which replaces the SpyCas9 PID.

2. The isolated, engineered protein of claim 1, having a PAM specificity of “NAA” or “NA” or “NAAN”.

3. The isolated, engineered protein of claim 2, further comprising at least one of the mutations R221K and N394K.

4. The isolated, engineered protein of claim 1, further comprising at least one of the mutations R221K and N394K.

5. The isolated, engineered protein of claim 1, wherein said SpyCas9 Protospacer Adjacent Motif (PAM) Interacting Domain (PID) amino acid sequence has 100% identity to the amino acid composition of the PID of the Streptococcus macacae Cas9 (Smac Cas9) protein comprising SEQ ID No: 33.

6. The isolated, engineered protein of claim 5, having a PAM specificity of “NAA” or “NA” or “NAAN”.

7. The isolated, engineered protein of claim 6, further comprising at least one of the mutations R221K and N394K.

8. The isolated, engineered protein of claim 5, further comprising at least one of the mutations R221K and N394K.

9. The isolated, engineered protein of claim 1, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

10. The isolated, engineered protein of claim 2, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

11. The isolated, engineered protein of claim 3, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

12. The isolated, engineered protein of claim 4, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

13. The isolated, engineered protein of claim 5, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

14. The isolated, engineered protein of claim 6, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

15. The isolated, engineered protein of claim 7, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

16. The isolated, engineered protein of claim 8, or a transgene expression thereof, in complex with a CRISPR system guide RNA that hybridizes with a target sequence, wherein the protein and the guide RNA do not naturally occur together, the protein and the guide RNA together forming an engineered, non-naturally occurring CRISPR-Cas system.

17. A method of altering expression of at least one gene product comprising:
introducing, into a eukaryotic cell containing and expressing a DNA molecule having a target sequence and encoding the gene product, an engineered, non-naturally occurring CRISPR-Cas system comprising one or more vectors comprising:
(a) a regulatory element operable in a eukaryotic cell operably linked to at least one nucleotide sequence encoding a CRISPR system guide RNA that hybridizes with the target sequence; and
(b) a second regulatory element operable in a eukaryotic cell operably linked to a nucleotide sequence encoding one or more proteins selected from the group consisting of:
(i) an isolated, engineered Streptococcus pyogenes Cas9 (SpyCas9) protein comprising SEQ ID No: 31 or SEQ ID No. 32, wherein said SpyCas9 is modified to have a Protospacer Adjacent Motif (PAM) Interacting Domain (PID) amino acid sequence that is identical to the amino acid composition of the PID of an isolated wild-type Streptococcus macacae Cas9 (Smac Cas9) protein comprising SEQ ID No: 33, which replaces the SpyCas9 PID, or a transgene expression thereof; and
(ii) an isolated, engineered Streptococcus pyogenes Cas9 (SpyCas9) protein comprising SEQ ID No: 31 or SEQ ID No. 32, wherein said SpyCas9 is modified to have a Protospacer Adjacent Motif (PAM) Interacting Domain (PID) amino acid sequence with at least 80% identity to the amino acid composition of the PID of an isolated wild-type Streptococcus macacae Cas9 (Smac Cas9) protein comprising SEQ ID No: 33, which replaces the SpyCas9 PID, or a transgene expression thereof;
wherein components (a) and (b) are located on same or different vectors of the system, whereby the guide RNA targets the target sequence and one or more of the proteins cleave the DNA molecule, whereby expression of the at least one gene product is altered; and,
wherein the proteins and the guide RNA do not naturally occur together.

18. The method of claim 17, wherein second regulatory element (b) has a PAM specificity of “NAA” or “NA” or “NAAN”.

19. The method of claim 18, wherein second regulatory element (b) further comprises at least one of the mutations R221K and N394K.

20. The method of claim 17, wherein second regulatory element (b) further comprises at least one of the mutations R221K and N394K.


SEQUENCE LISTING (33 sequences)

SEQ ID NO: 1 - RNA, 72 nt, synthetic construct
  misc_feature 1..72: note = ribonucleprotein complex enzyme
  source 1..72: mol_type = other RNA
rcrgrararargrgrtrtrtrtrgrcrarcrtrcrgrarcrgrtrtrtrtrargrargrcrtrartrgrcrt

SEQ ID NO: 2 - RNA, 132 nt, Streptococcus pyogenes
  source 1..132: mol_type = other RNA
rargrcrartrargrcrarargrtrtrarararartrarargrgrcrtrargrtrcrcrgrtrtrartrcrararcrtrtrgrararararargrtrgrgrcrarcrcrgrargrtrcrgrgrtrgrcrtrt

SEQ ID NO: 3 - DNA, 39 bp, unidentified
  misc_feature 1..39: note = derived from 5'-NNNNNNNN-3' library
  misc_feature 21..24: note = n is a, c, g, or t
  source 1..39: mol_type = genomic DNA
cgaaaggttttgcactcgacnnnnaccaacgaaagggcc

SEQ ID NO: 4 - DNA, 88 bp, synthetic construct
  misc_feature 1..88: note = sgRNA for Cas9
  source 1..88: mol_type = other DNA
gttttagagctatgctggaaacagcatagcaagttaaaataaggctagtccgttatcaacttgaaaaagtggcaccgagtcggtgctt

SEQ ID NO: 5 - DNA, 20 bp, synthetic construct
  misc_feature 1..20: note = gRNA for AsCas12a
  source 1..20: mol_type = other DNA
taatttctactcttgtagat

SEQ ID NO: 6 - DNA, 20 bp, synthetic construct
  misc_feature 1..20: note = gRNA for LbCas12a
  source 1..20: mol_type = other DNA
aatttctactaagtgtagat

SEQ ID NO: 7 - DNA, 20 bp, unidentified
  misc_feature 1..20: note = Target for CAAATTCC PAM w/ Cas9
  source 1..20: mol_type = genomic DNA
gaacccggatcaatgaatat

SEQ ID NO: 8 - DNA, 20 bp, unidentified
  misc_feature 1..20: note = Target for CAAATTCC PAM w/ Cas12a
  source 1..20: mol_type = genomic DNA
atattcattgatccgggttc

SEQ ID NO: 9 - DNA, 20 bp, unidentified
  misc_feature 1..20: note = Target for CAACCCCA PAM w/ Cas9
  source 1..20: mol_type = genomic DNA
gctccccgctccaacaccct

SEQ ID NO: 10 - DNA, 20 bp, unidentified
  misc_feature 1..20: note = Target for CAACCCCA PAM w/ Cas12a
  source 1..20: mol_type = genomic DNA
agggtgttggagcggggagc

SEQ ID NO: 11 - DNA, 20 bp, unidentified
  misc_feature 1..20: note = Target for CAAGCCGT PAM w/ Cas9
  source 1..20: mol_type = genomic DNA
gggaagtagagcaatctccc

SEQ ID NO: 12 - DNA, 20 bp, unidentified
  misc_feature 1..20: note = Target for CAAGCCGT PAM w/ Cas12a
  source 1..20: mol_type = genomic DNA
gggagattgctctacttccc

SEQ ID NO: 13 - DNA, 20 bp, unidentified
  misc_feature 1..20: note = Target for CAATGTGC PAM w/ Cas9
  source 1..20: mol_type = genomic DNA
gccacagtgtgtccctctga

SEQ ID NO: 14 - DNA, 20 bp, unidentified
  misc_feature 1..20: note = Target for CAATGTGC PAM w/ Cas12a
  source 1..20: mol_type = genomic DNA
tcagagggacacactgtggc

SEQ ID NO: 15 - DNA, 20 bp, unidentified
  misc_feature 1..20: note = Target for TAACCTCA PAM w/ Cas9
  source 1..20: mol_type = genomic DNA
gctcaggccctgtccgcacg

SEQ ID NO: 16 - DNA, 20 bp, unidentified
  misc_feature 1..20: note = Target for TAACCTCA PAM w/ Cas12a
  source 1..20: mol_type = genomic DNA
cgtgcggacagggcctgagc

SEQ ID NO: 17 - DNA, 20 bp, unidentified
  misc_feature 1..20: note = Target for TAAGGCCC PAM w/ Cas9
  source 1..20: mol_type = genomic DNA
gttccatcggtatggtgtcc

SEQ ID NO: 18 - DNA, 20 bp, unidentified
  misc_feature 1..20: note = Target for TAAGGCCC PAM w/ Cas12a
  source 1..20: mol_type = genomic DNA
ggacaccataccgatggaac

SEQ ID NO: 19 - DNA, 20 bp, unidentified
  misc_feature 1..20: note = Target for GAAGTCGA PAM w/ Cas9
  source 1..20: mol_type = genomic DNA
ggtagcaagagctccagaga

SEQ ID NO: 20 - DNA, 20 bp, unidentified
  misc_feature 1..20: note = Target for GAAGTCGA PAM w/ Cas12a
  source 1..20: mol_type = genomic DNA
tctctggagctcttgctacc

SEQ ID NO: 21 - DNA, 20 bp, unidentified
  misc_feature 1..20: note = Target for GAAAGTGA PAM w/ Cas9
  source 1..20: mol_type = genomic DNA
gattggcgaggagggagcag

SEQ ID NO: 22 - DNA, 20 bp, unidentified
  misc_feature 1..20: note = Target for GAAAGTGA PAM w/ Cas12a
  source 1..20: mol_type = genomic DNA
ctgctccctcctcgccaatc

SEQ ID NO: 23 - DNA, 20 bp, unidentified
  misc_feature 1..20: note = Target for GAAACCAG PAM w/ Cas9
  source 1..20: mol_type = genomic DNA
gcctggaaatagccaggtca

SEQ ID NO: 24 - DNA, 20 bp, unidentified
  misc_feature 1..20: note = Target for GAAACCAG PAM w/ Cas12a
  source 1..20: mol_type = genomic DNA
tgacctggctatttccaggc

SEQ ID NO: 25 - DNA, 20 bp, unidentified
  misc_feature 1..20: note = Target for AAACCAGC PAM w/ Cas9
  source 1..20: mol_type = genomic DNA
gctggaaatagccaggtcag

SEQ ID NO: 26 - DNA, 20 bp, unidentified
  misc_feature 1..20: note = Target for AAACCAGC PAM w/ Cas12a
  source 1..20: mol_type = genomic DNA
ctgacctggctatttccagc

SEQ ID NO: 27 - DNA, 20 bp, unidentified
  misc_feature 1..20: note = Target for AAAGTGAG PAM w/ Cas9
  source 1..20: mol_type = genomic DNA
gttggcgaggagggagcagg

SEQ ID NO: 28 - DNA, 20 bp, unidentified
  misc_feature 1..20: note = Target for AAAGTGAG PAM w/ Cas12a
  source 1..20: mol_type = genomic DNA
cctgctccctcctcgccaac

SEQ ID NO: 29 - DNA, 20 bp, unidentified
  misc_feature 1..20: note = Target for AAATTCCA PAM w/ Cas9
  source 1..20: mol_type = genomic DNA
gacccggatcaatgaatatc

SEQ ID NO: 30 - DNA, 20 bp, unidentified
  misc_feature 1..20: note = Target for AAATTCCA PAM w/ Cas12a
  source 1..20: mol_type = genomic DNA
gatattcattgatccgggtc

SEQ ID NO: 31 - protein, 289 aa, Streptococcus pyogenes
FATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 32 - protein, 289 aa, Streptococcus pyogenes
FATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADAQLDKVLSAYNKHRDKPIREQAENIIHLFTLTNLGAPAAFKYFDTTFKQKQYRSTKEVLDATLIHQSITGLYETRIDLSQLGGD

SEQ ID NO: 33 - protein, 281 aa, Streptococcus macacae
ISNIKRVLSYPQVNIVKKVEIQTVGQNGGLFDDNPKSPLEVTPSKLVPLKKELNPKKYGGYQKPTTAYPVLLITDTKQLIPISVMNKKQFEQNPVKFLRDRGYQQVGKNDFIKLPKYTLVDIGDGIKRLWASSKEIHKGNQLVVSKKSQILLYHAHHLDSDLSNDYLQNHNQQFDVLFNEIISFSKKCKLGKEHIQKIENVYSNKKNSASIEELAESFIKLLGFTQLGATSPFNFLGVKLNQKQYKGKKDYILPCTEGTLIRQSITGLYETRVDLSKIGED